(19) United States
(12) Patent Application Publication
(10) Pub. No.: US 2017/0260276 A1
(43) Pub. Date: Sep. 14, 2017

(54) TREATMENT FOR RHEUMATOID ARTHRITIS

(71) Applicant: MEDIMMUNE LIMITED, CAMBRIDGE (GB)

(72) Inventors: DAVID CLOSE, CAMBRIDGE (GB); ALEX GODWOOD, Cambridge (GB); MARK HOPTON, CAMBRIDGE (GB)

(21) Appl. No.: 15/310,226

(22) PCT Filed: May 18, 2015
(86) PCT No.: PCT/EP2015/060902

§ 371 Date: Nov. 10, 2016

Related U.S. Application Data
(60) Provisional application No. 62/000,235, filed on May 19, 2014.

(51) Int. Cl. C07K 16/28; A61K 39/395; A61K 31/519

(52) U.S. Cl. CPC C07K 16/2866; A61K 31/519; A61K 39/3955; A61K 2039/545

(57) ABSTRACT

Treatment of rheumatoid arthritis (RA) to provide clinical benefit in patients, including decrease in DAS28-CRP by more than 1.2 and/or improvement determined by ACR20, ACR50 or ACR70, comprising administering therapeutic antibody mavrilimumab.

This disclosure relates to treating rheumatoid arthritis by inhibiting biological effects of granulocyte/macrophage colony stimulator factor receptor alpha subunit (GM-CSFRα), by administering an inhibitor such as the therapeutic antibody mavrilimumab.

### BACKGROUND

Rheumatoid arthritis (RA) is a chronic inflammatory and destructive joint disease that affects approximately 1% of the population in the industrialized world. It affects approximately 3 times more women than men, and onset is generally between 40-60 years of age. RA is characterised by hyperplasia and inflammation of the synovial membrane, inflammation within the synovial fluid, and progressive destruction of the surrounding bone and cartilage. It is a painful condition, can cause severe disability and ultimately affects a person's ability to carry out everyday tasks. Effects of RA vary between individuals, but the disease can progress very rapidly, causing swelling and damaging cartilage and bone around the joints. Any joint can be affected but it is commonly the hands, feet and wrists. Internal organs such as the lungs, heart and eyes can also be affected.

The cause of RA remains unknown, although studies have elucidated some aspects of the inflammatory processes underlying the disease. RA is believed to be initiated and driven through a T-cell mediated, antigen-specific process. In brief, the presence of an unidentified antigen in a susceptible host is thought to initiate a T-cell response that leads to the production of T-cell cytokines with consequent recruitment of inflammatory cells, including neutrophils, macrophages, and B-cells.

Many pro- and anti-inflammatory cytokines are produced in the rheumatoid joint. Disease progression, reactivation and silencing are mediated via dynamic changes in cytokine production within the joint. In particular, TNF-α and IL-1 are considered to exert pivotal roles in the pathogenesis of RA.

GM-CSF is a type 1 pro-inflammatory cytokine believed to contribute to the pathogenesis of RA through the activation, differentiation and survival of neutrophils and macrophages. Studies in rodent models have suggested a central and non-redundant role for GM-CSF in the development and progression of RA (CAMPBELL, I. K., et al. (1997) Annal Res Dis, 56, 364-368; BISCHOF, R. J., et al. (2000) Clin Exp Immunol, 119, 361 -367; Campbell, I. K., M. J. Rich, et al. (1998). J Immunol 161 (7): 3639-44; Hamilton, J. A. (2002). Trends Immunol 23(8): 403-8; YANG, Y. H. AND J. A. HAMILTON (2001) Arthritis Rheumatol, 44, 1 1 1 -1 19). For example, in collagen induced arthritis (CIA) and monoarticular arthritis models in mice, administration of murine anti-GM-CSF monoclonal antibody (mAb) significantly ameliorated disease severity. In the CIA model, mAb treatment was effective in treating progression of established disease, histopathology and significantly lowering joint IL-1 and TNF-α levels.

Mavrilimumab (CAM-3001) is a human monoclonal antibody targeting the alpha subunit of GM-CSFR (GM-CSFRα). The structure of the mavrilimumab mAb is described in PCT Publication No. WO/2007/110631, incorporated herein by reference. Phase 1 single ascending intravenous dose study of mavrilimumab in 32 subjects with RA showed an adequate safety and tolerability profile, and initial indications of biologic activity, such as normalization of acute phase reactants and possible reductions in Disease Activity Score 28-joint assessment (DAS28) in patients with moderate disease activity (Burmester et al. Annals of the Rheumatic Diseases 70: 1542-1549 2011). A Phase IIa trial was also recently completed (see PCT Publication No. WO 2013/053767, incorporated herein by reference).

The current drug management of RA includes palliative treatment, particularly analgesics and non-steroidal anti-inflammatory drugs (NSAIDs), and treatment to limit disease severity and progression, including disease modifying drugs (DMARDs) and biologics. The established management of RA using DMARDs includes the administration of single DMARDs, e.g. methotrexate, sulfasalazine, hydroxychloroquine or leflunomide, and their use in combination, for example methotrexate can be combined with sulfasalazine and/or hydoxychloroquine. Methotrexate is an antimetabolite and antifolate, although its efficacy in RA is believed to be due to the suppression of T cell activation and expression of adhesion molecule (ICAM-1) (Johnston et al. Clin Immunol. 1 14(2): 154-163 2005). Clinical use of biologic agents for RA mainly involves inhibitors of TNF-α. These include infliximab (Remicade®), etanercept (Enbrel®), adalimumab (Humira®), certolizumab pegol (Cimzia®)) and golimumab (Simponi®). Infliximab is given by intravenous infusion whereas the other four are injected subcutaneously at home by the patient. An anti-interleukin 1 inhibitor, Kineret®, has also been developed. More recently, the anti-B lymphocyte drug rituximab (Mabthera® or Rituxan®) has been approved for treatment of RA patients who have failed anti-TNF therapy. Mabthera® is given as an initial treatment of two infusions 14 days apart. Those patients who experience improvement lasting up to six months can then have repeat infusions. Despite these advances, RA represents a significant unmet medical need. Although early diagnosis and treatment can improve the long term prognosis, there is currently no cure for RA. Improved therapies are needed to reduce the severity and progression of the disease and to improve the quality of life of patients.

One measure of how well RA is being controlled is the Disease Activity Score (DAS) (Fransen & van Riel Clin Exp Rheumatol 23:S93-S99 2005). The DAS is calculated by a medical practitioner based on various validated measures of disease activity, including physical symptoms of RA. A reduction in DAS reflects a reduction in disease severity. A DAS of less than 2.6 indicates disease remission. DAS between 2.6 and 3.2 indicates low disease activity. A DAS greater than 3.2 indicates increased disease activity and at this level a patient's therapy could be reviewed to determine whether a change in therapy is warranted. DAS greater than 5.1 indicates severe disease activity. Variations in calculating DAS can include assessing different numbers of joints in the patient and monitoring different blood components. DAS28 is the Disease Activity Score in which 28 joints in the body are assessed to determine the number of tender joints and the number of swollen joints (Prevoo et al. Arthritis Rheum 38:44-48 1995). When the DAS28 calculation includes a measurement of C-reactive protein (CRP) rather than erythrocyte sedimentation rate (ESR), it is referred to as DAS28-CRP (Smolen et al. Rheumatology 42:244-257 2003; Wells G, et al. Annals of the Rheumatic Diseases 68: 954-960 2009). CRP is believed to be a more direct measure of inflammation than ESR, and is more sensitive to short term changes (Kushner, Arthritis Rheum 34: 1065-68 1991). CRP production is associated with radiological progression in RA (van Leeuwen M A, et al. Br J Rheumatol 32(suppl 3):9-13 1993) and is considered at least as valid as ESR to measure RA disease activity (Mallya R K, et al. J Rheumatol 9:224-8 1982; Wolfe F. J Rheumatol 24:1477-85 1997).

The American College of Rheumatology (ACR) proposed a set of criteria for classifying RA. The commonly used criteria are the ACR 1987 revised criteria (Arnett et al. Arthritis Rheum. 31:315-324 1988). Diagnosis of RA according to the ACR criteria requires a patient to satisfy a minimum number of listed criteria, such as tender or swollen joint counts, stiffness, pain, radiographic indications and measurement of serum rheumatoid factor. ACR 20, ACR 50 and ACR 70 are commonly used measures to express efficacy of RA therapy, particularly in clinical trials. ACR 20 represents a 20% improvement in the measured ACR criteria. Analogously, ACR 50 represents a 50% improvement in the measured ACR criteria, and ACR 70 represents a represents a 70% improvement in the measured ACR criteria.

An individual, patient reported measure of disability in RA patients is the Health Assessment Questionnaire Disability Index (HAQ-DI). HAQ-DI scores represent physical function in terms of the patient's reported ability to perform everyday tasks, including the level of difficulty they experience in carrying out the activity. By recording patients' ability to perform everyday activities, the HAQ-DI score can be used as one measure of their quality of life.

### SUMMARY

This disclosure provides treatments for RA to provide clinical benefit including reducing DAS28-CRP and increasing the number of patients who obtain clinical benefit as determined by ACR 20, ACR 50 and ACR 70. Further, the disclosure relates to methods and compositions for improving physical function of RA patients, as determined by the HAQ-DI.

Reported here are significant positive results from a Phase 2 clinical trial in which RA patients received the anti-GM-CSFRα antibody mavrilimumab. In this double blind trial, RA patients with at least moderate disease activity according to DAS28-CRP and who were already undergoing treatment with stable doses of methotrexate were randomized to varying subcutaneous doses of mavrilimumab or placebo. No changes in respiratory function parameters, opportunistic infections, serious hypersensitivity reactions or laboratory abnormalities were observed in this study over the treatment period or during a 12 week follow up period, indicating a good safety profile.

Mavrilimumab is a human IgG4 monoclonal antibody designed to modulate macrophage activation, differentiation and survival by targeting the GM-CSFRα. It is a potent neutralizer of the biological activity of GM-CSFRα and, without wishing to be bound by theory, can exert therapeutic effects by binding GM-CSFRα on leukocytes within the synovial joints of RA patients, leading to reduced cell survival and activation. WO2007/110631 reports the isolation and characterization of mavrilimumab and variants of it which share an ability to neutralize the biological activity of GM-CSFRα with high potency. The functional properties of these antibodies are believed to be attributable, at least in part, to binding a Tyr-Leu-Asp-Phe-Gln motif at positions 226 to 230 of human GM-CSFRα, thereby inhibiting association between GM-CSFRα and its ligand GM-CSF.

One aspect of the disclosure is drawn to a method of treating rheumatoid arthritis, comprising administering to a patient or a population of patients in need of treatment a composition comprising mavrilimumab. In certain embodiments, the composition comprising mavrilimumab is administered at a dose of 150 mg mavrilimumab. In certain embodiments, the 150 mg mavrilimumab are administered every two weeks. In certain embodiments, mavrilimumab is administered orally, intravenously, or by subcutaneous administration. In certain embodiments, 150 mg mavrilimumab are administered subcutaneously every two weeks. In certain embodiments, the method can decrease DAS28-CRP from baseline by more than 1.7, more than 1.8, more than 1.9, more than 2.0, or more than 2.1 in one or more of the patients. In certain embodiments, the method can improve at least 20% treatment efficacy (ACR 20), at least 50% treatment efficacy (ACR50), or at least 70% treatment efficacy (ACR70) as determined by the 1987 American College of Rheumatology (ACR) criteria in one or more of the patients. Where DAS28-CRP is decreased from baseline by more than 1.7, by more than 1.8, or by more than 1.9 in one or more of the patients, in certain embodiment the decrease is within 169 days of initiation of treatment, or is within 85 days of initiation of treatment. Where DAS28-CRP is decreased from baseline by more than 2.0 or by more than 2.1 in one or more treated patients, in certain embodiments the decrease is within 169 days of initiation of treatment. In certain embodiments, the method can achieve at least 20% treatment efficacy (ACR 20) in one or more of the patients as determined by the 1987 American College of Rheumatology (ACR) criteria. Where ACR 20 is achieved, in certain embodiments it is achieved within 170 days, 169 days, 168 days, or any integer of days thereof after initiation of treatment. ACR 20 can be achieved within 169 days, 85 days, 42 days, or 14 days of initiation of treatment. In certain embodiments, the method can achieve at least 50% treatment efficacy (ACR 50) in one or more of the patients as determined by the 1987 American College of Rheumatology (ACR) criteria. Where ACR 50 is achieved, in certain embodiments it is achieved within 169 days, 85 days, 42 days, or 14 days of initiation of treatment. In certain embodiments, the method can achieve at least 70% treatment efficacy (ACR 70) in one or more of the patients as determined by the 1987 American College of Rheumatology (ACR) criteria. Where ACR 70 is achieved, in certain embodiments it is achieved within 160 days, 80 days, 40 days, or 14 days of initiation of treatment. ACR 70 can be achieved within 170 days, 169 days, 168, days, or any integer of days thereof up to and including 14 days.

Certain embodiments can result in remission or reduced time to onset of remission of rheumatoid arthritis symptoms in one or more of the patients. In certain embodiments, such remission of rheumatoid arthritis symptoms is in at least 10%, at least 15%, or at least 20% of the patients. Certain embodiments can achieve ACR 20 in at least 60%, or at least 65%, or at least 70% of the patients within 113 days. Certain embodiments can achieve ACR 50 in at least 15%, or at least 20%, or at least 25% of the patients within 113 days. In some embodiments, at least 30%, or at least 35%, or at least 40% of the patients achieved ACR 50 within 169 days. In certain embodiments at least 10%, or at least 13% of the patients can achieve ACR 70 within 113 days. In certain embodiments, ACR20, ACR50, or ACR70 is achieved within 85 days of initiation of treatment in one or more of the patients. Certain embodiments can improve physical function in one or more of the patients, as determined by HAQ-DI.

Certain aspects of the disclosure are drawn to a method for improving physical function of an RA patient or a population of RA patients, as determined by a HAQ-DI score, comprising administering to a patient or population of patients in need of treatment a composition comprising mavrilimumab, wherein the composition is administered at a dose of 150 mg. In certain embodiments, mavrilimumab is administered every two weeks. In certain embodiments mavrilimumab is administered orally, intravenously, or by subcutaneous administration. In certain embodiments, the method can improve the HAQ-DI score by at least 0.25 in one or more of the patients. In certain embodiments, the method can improve the HAQ-DI score by at least 0.25 in at least 60% of the patients. In certain embodiments, the improvement in HAQ-DI is achieved within six weeks of initiation of treatment.

Certain aspects of the disclosure are drawn to a method of inducing remission of RA in a patient or a population of patients as measured by a DAS28-CRP of less than 2.6, comprising administering to a patient or a population of patients in need of treatment a composition comprising mavrilimumab, wherein the composition is administered at a dose of 150 mg. In certain embodiments, mavrilimumab is administered every two weeks. In certain embodiments, mavrilimumab is administered orally, intravenously, or subcutaneously. In certain embodiments, mavrilimumab is administered at 150 mg, every two weeks, by subcutaneous administration. In certain embodiments, the onset of remission is achieved within 170 days, or within 805 days, or within 40 days, or within 14 days of initiation of treatment. In certain embodiments, the onset of remission is achieved within 170 days, 169 days, 168 days, or any integer of days thereof after initiation of treatment.

In any of the aspects, the method further comprises administering an additional therapeutic agent. In certain embodiments, the additional therapeutic agent comprises a disease modifying anti-rheumatic drug (DMARD). In certain embodiments, the additional therapeutic agent is methotrexate. In certain embodiments, the methotrexate is administered at a dose of 7.5 to 25 mg per week. In certain embodiments, a stable dose of methotrexate is administered for at least 4 weeks prior to administration of the composition comprising mavrilimumab. In certain embodiments, the method comprises administering the composition comprising mavrilimumab to the patient in combination with continued doses of methotrexate.

One or more of the patients may have a baseline DAS28-CRP of at least 3.2 prior to treatment. In certain embodiments, one or more of the patients has a baseline DAS28-CRP greater than 5.1 prior to treatment. One or more of the patients may test positive for rheumatoid factor and/or anti-cyclic citrullinated peptide (CCP) IgG antibodies prior to treatment. One or more of the patients may not have medically significant respiratory disease.

Certain aspects of the disclosure are drawn to a composition comprising 150 mg of mavrilimumab. The composition is suitable for oral consumption, intravenous administration, or subcutaneous administration, for use according to any of the preceding methods. In certain embodiments, the composition comprises 150 mg of mavrilimumab for subcutaneous administration. In certain embodiments, the composition is formulated for administration in combination with methotrexate.

Certain aspects of the disclosure are drawn to a method of treating rheumatoid arthritis comprising administering to a patient or a population of patients in need of treatment a composition comprising mavrilimumab, wherein the composition is administered at a dose of 150 mg mavrilimumab every two weeks by subcutaneous administration, and wherein the treatment can result in an increase in duration of DAS28-CRP remission. In certain embodiments, the duration of DAS28-CRP remission achieved is at least 60 days in at least 60% of patients. In certain embodiments, the duration of DAS28-CRP remission achieved is at least 80 days in at least 50% of patients. In certain embodiments, the duration of DAS28-CRP remission achieved is at least 130 days in at least 25% of patients.

A patient to be treated may have RA as determined according to the 1987 ACR criteria. The patient may test positive for rheumatoid factor (RF) and/or anti-cyclic citrullinated peptide (CCP) IgG antibodies prior to treatment. RF positive and anti-CCP antibody positive status confirm diagnosis of RA. The patient may have had RA for a duration of at least 5 years or at least 7 years, for example between 5 and 10 years.

In certain embodiments, patients who are to be treated with mavrilimumab according to the disclosure do not have respiratory disease. Patients can be tested prior to administration of mavrilimumab to confirm that they do not have medically significant respiratory disease, e.g. pneumonitis. Methods of testing for respiratory disease include chest x-ray, and assessment of pulmonary function by spirometry and diffusing capacity for carbon monoxide (DLCO). In certain embodiments, patients also do not have clinically significant chronic or recurrent infection, such as hepatitis C or chronic active hepatitis B infection. Patients can be tested for such infection prior to treatment as described herein.

Where treatment and clinical benefit are described here with reference to “a patient”, it will be appreciated that this can include treatment of a group of patients. In certain embodiments, patients are human adults. Patients can for example be aged from 18 to 80 years old.

Clinical benefit achieved in the methods described herein can comprise any one or more of the following outcomes.

The clinical benefit can be a decrease in DAS28-CRP by more than 1.2. The reduction in DAS28-CRP can be achieved in at least 40%, at least 50% or at least 60% of patients treated. The clinical benefit can comprise an increasing the proportion of patients who achieve a decrease in DAS28-CRP by more than 1.2, compared with control patients who are not treated.

The clinical benefit can comprise remission of RA. Typically, remission is defined by a DAS28-CRP of less than 2.6. Remission can be achieved in at least 10% or patients, or at least 20% of patients. In patients treated as described herein, the time to onset of remission can be reduced compared with patients who are not treated as described herein. Time to remission can be reduced by approximately 50%.

The clinical benefit can be an improvement of at least 20%, at least 50% or at least 70% treatment efficacy as determined by the 1987 ACR criteria, i.e. the clinical benefit can be achieving ACR 20, ACR 50 or ACR 70, respectively. In certain embodiments, the clinical benefit comprises achieving ACR 20 in at least 40, 50, 60 or 70% of patients. It can comprise achieving ACR 50 in at least 20% or at least 30% of patients. It can comprise achieving ACR 70 in at least 5%, 10% or 15% of patients.

A form of clinical benefit that is of particular value to RA patients is an improvement in their ability to perform everyday activities. Methods of the disclosure can comprise improvement in the patient's self-assessed disability measured by the Health Assessment Questionnaire, known as HAQ-DI. Methods comprising providing clinical benefit to an RA patient, wherein the clinical benefit comprises improving physical function of an RA patient as determined by HAQ-DI, and compositions and kits for use in such methods, are all aspects of the disclosure. Clinical benefit can comprise improving physical function of an RA patient as determined by HAQ-DI. In certain embodiments, a statistically significant improvement in HAQ-DI is achieved within twelve, ten, eight or six weeks of starting treatment according to the disclosure, or within four weeks, or within two weeks. The improvement can be at least a 0.25 improvement in HAQ-DI, i.e. a reduction of 0.25 or more in the patient's HAQ-DI score. In certain embodiments, the improvement is at least a 0.30, 0.40 or 0.45 improvement in HAQ-DI score. Improvement is generally measured with reference to the patient's baseline average HAQ-DI score prior to treatment with an inhibitor according to the disclosure. Where a group of patients is treated, the improvement can be observed in at least 50%, at least 60% or at least 70% of treated patients.

The clinical benefit can be achieved sooner in treated patients compared with patients who are not treated according to the disclosure. For example, patients who are treated according to the disclosure, with mavrilimumab in combination with methotrexate can achieve clinical benefit sooner than patients treated with methotrexate alone. The time to onset of response, or period of treatment before the clinical benefit is achieved, can be decreased by at least 10%, at least 20%, at least 30%, at least 40% or at least 50% in patients treated with the combination compared with patients who are treated with methotrexate alone. In certain embodiments, the clinical benefit is achieved within 85 days. So, for example, DAS28-CRP can be decreased by more than 1.2 within 85 days. In certain embodiments, the onset of response occurs within 2 weeks. Thus, clinical benefit can be achieved within 14 days of treatment with mavrilimumab.

Patients can be monitored during and/or following a course of treatment with mavrilimumab, to assess the level of clinical benefit, for example by measuring DAS28-CRP and/or determining clinical benefit according to the ACR criteria and/or measuring HAQ-DI. The method can comprise determining that the clinical benefit is achieved, e.g. that the specified reduction in DAS28-CRP, and/or achievement of ACR 20, ACR 50 or ACR 70 is met, and/or that the HAQ-DI score is improved, as discussed elsewhere herein.

In certain embodiments, doses are administered at intervals of 14 days (i.e. on day 1, day 15, day 29, etc). Alternatively, doses can be administered at intervals of 28 days. Further details of possible dosages and administration are described elsewhere herein. The method can comprise administering mavrilimumab to the patient, by doses at intervals of 14 days, for a duration of at least 85 days although treatment can be continued beyond 85 days, and patients can be maintained on the treatment indefinitely provided that they are suitably monitored. In certain embodiments, clinical benefit is achieved by day 85, and in certain embodiments by day 14, of the treatment. In certain embodiments clinical benefit is achieved after only a single dose, or after only two doses, of treatment according to the disclosure.

Mavrilimumab can be administered by any suitable method. Typical methods for antibody administration are oral, subcutaneous or intravenous delivery. In certain embodiments, a composition comprising mavrilimumab is formulated for subcutaneous or intravenous administration.

Aspects of treating RA can comprise administering a composition comprising an inhibitor according to the disclosure to the patient in combination with one or more additional therapeutic agents. Additional therapeutic agents can comprise any one or more of the following: analgesics; NSAIDs; steroids; DMARDs for the ‘treatment of RA’ e.g. methotrexate, sulfasalazine, hydoxychloroquine, leflunomide. Biologic DMARDs include TNF-α inhibitors e.g. infliximab (Remicade®); etanercept (Enbrel®), adalimumab (Humira®), certolizumab pegol (Cimzia®), golimumab (Simponi®), IL-1 inhibitors e.g. Kineret®, and anti-B lymphocyte agents e.g. Rituximab, abatacept (Humira®) or toclizumab.

### BRIEF DESCRIPTION OF THE DRAWINGS/FIGURES

FIG. 1A shows an outline of the clinical study design.

FIG. 1B shows the distribution of patients within the 30 mg mavrilimumab, 100 mg mavrilimumab, 150 mg mavrilimumab, and placebo cohorts.

FIG. 2 shows DAS28 adjusted mean change from baseline (+/−SE) from the beginning of mavrilimumab administration until approximately 24 weeks (Day 169).

FIG. 3A shows a comparison of 30 mg mavrilimumab, 100 mg mavrilimumab, 150 mg mavrilimumab, and placebo treatment in achievement of ACR20, ACR50, and ACR70 at Day 169.

FIG. 3B shows achievement of ACR20 by patients treated with 30 mg mavrilimumab, 100 mg mavrilimumab, 150 mg mavrilimumab, and placebo by day from start of treatment to Day 169.

FIG. 3C shows achievement of ACR50 by patients treated with 30 mg mavrilimumab, 100 mg mavrilimumab, 150 mg mavrilimumab, and placebo by day from start of treatment to Day 169.

FIG. 3D shows achievement of ACR70 by patients treated with 30 mg mavrilimumab, 100 mg mavrilimumab, 150 mg mavrilimumab, and placebo by day from start of treatment to Day 169.

FIG. 4A shows DAS28-CRP remission (<2.6) rate for each mavrilimumab treatment group and placebo in the clinical trial by day from start of treatment to Day 169.

FIG. 4B shows the time to onset of DAS28-CRP remission for each mavrilimumab treatment group and placebo in the clinical trial.

FIG. 4C shows the duration of DAS28-CRP remission for each mavrilimumab treatment group and placebo in the clinical trial.

FIG. 4D shows the time to onset of DAS28-CRP low disease activity for each mavrilimumab treatment group and placebo in the clinical trial.

FIG. 4E shows the duration of DAS28-CRP low disease activity for each mavrilimumab treatment group and placebo in the clinical trial.

FIG. 5A shows the HAQ-DI adjusted mean change from baseline (+/−SE) for each mavrilimumab treatment group and placebo in the clinical trial by day from start of treatment to Day 169.

FIG. 5B shows HAQ-DI responders (improvement>=0.25) for each mavrilimumab treatment group and placebo in the clinical trial at Day 169.

FIG. 6A shows the swollen joint count adjusted mean change from baseline (+/−SE) for each mavrilimumab treatment group and placebo in the clinical trial by day from start of treatment to Day 169.

FIG. 6B shows the tender joint count adjusted mean change from baseline (+/−SE) for each mavrilimumab treatment group and placebo in the clinical trial by day from start of treatment to Day 169.

FIG. 6C shows the Patient Global Assessment adjusted mean change from baseline (+/−SE) for each mavrilimumab treatment group and placebo in the clinical trial by day from start of treatment to Day 169.

FIG. 6D shows the Patient Pain adjusted mean change from baseline (+/−SE) for each mavrilimumab treatment group and placebo in the clinical trial by day from start of treatment to Day 169.

FIG. 6E shows the Physician Global Assessment adjusted mean change from baseline (+/−SE) for each mavrilimumab treatment group and placebo in the clinical trial by day from start of treatment to Day 169.

FIG. 6F shows the CPR adjusted geometric mean ratio to baseline (+/−SE) for each mavrilimumab treatment group and placebo in the clinical trial by day from start of treatment to Day 169.

FIG. 7A shows FACIT-fatigue responders (improvement>=3) at Day 169 for each mavrilimumab treatment group and placebo in the clinical trial.

FIG. 7B shows SF-36® Physical Component Summary (PCS) responders (improvement>=3.1) at Day 169 for each mavrilimumab treatment group and placebo in the clinical trial.

FIG. 7C shows SF-36® Mental Component Summary (MCS) responders (improvement>=3.8) at Day 169 for each mavrilimumab treatment group and placebo in the clinical trial.

FIG. 8 shows the clinical trial data with Demin longitudinal meta-analysis showing treatment with mavrilimumab compared with key competitors.

FIG. 9A shows an outline of the clinical study design of Japanese subjects.

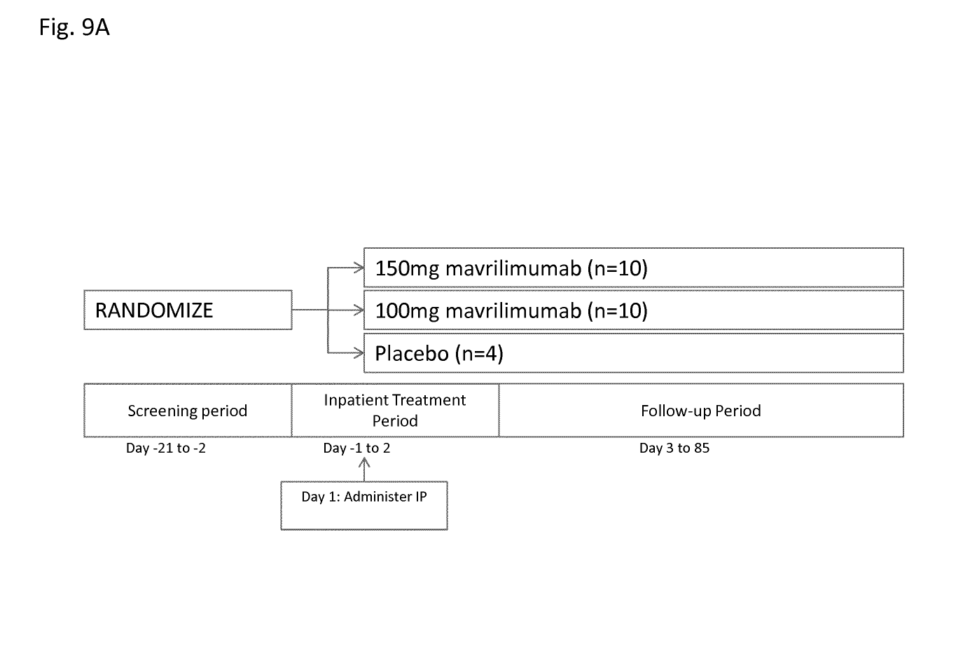

FIG. 9B shows the distribution of patients within the 100 mg mavrilimumab, 150 mg mavrilimumab, and placebo cohorts of the Japanese subject study.

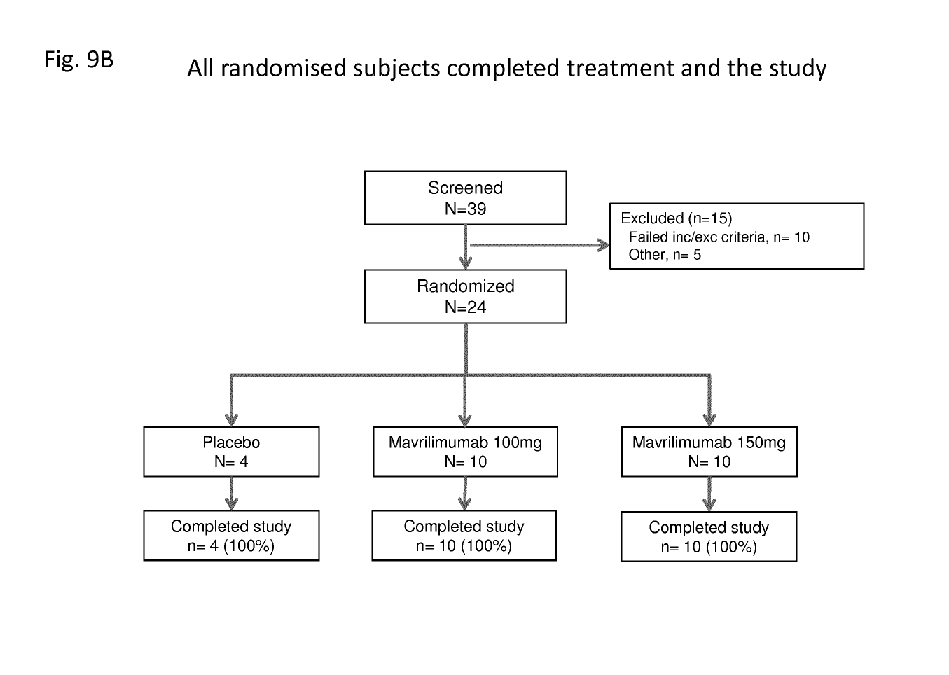

FIG. 10 shows the mean pharmacokinetic profiles of single SC mavrilimumab doses (100 mg and 150 mg) in healthy Japanese subjects.

FIG. 11A shows a cross-study comparison of pharmacokinetic results between trials.

FIG. 11B shows a cross-study comparison of pharmacokinetic results between trials.

FIG. 11C shows a cross-study comparison of pharmacokinetic results between trials.

FIG. 12A shows that the observed PK profiles of subjects in the Japanese trial fell within the predicted range at 100 mg mavrilimumab.

FIG. 12B shows that the observed PK profiles of subjects in the Japanese trial fell within the predicted range at 150 mg mavrilimumab.

FIG. 13A shows neutrophil profiles of subjects with a value less than lower limit of normal (LLN).

FIG. 13B shows alanine amino transferase (ALT) profiles of subjects with a value greater than upper limit of normal (ULN).

### DETAILED DESCRIPTION

### Definitions

It is to be noted that the term “a” or “an” entity refers to one or more of that entity; for example, “an anti-IL-5α antibody” is understood to represent one or more anti-IL-5α antibodies. As such, the terms “a” (or “an”), “one or more,” and “at least one” can be used interchangeably herein.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “antibody” (or a fragment, variant, or derivative thereof) refers to at least the minimal portion of an antibody which is capable of binding to antigen, e.g., at least the variable domain of a heavy chain (VH) and the variable domain of a light chain (VL) in the context of a typical antibody produced by a B cell. Basic antibody structures in vertebrate systems are relatively well understood. See, e.g., Harlow et al., Antibodies: A Laboratory Manual, (Cold Spring Harbor Laboratory Press, 2nd ed. 1988).

Antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is generally meant that an antibody or fragment, variant, or derivative thereof binds to an epitope via its antigen-binding domain, and that the binding entails some complementarity between the antigen binding domain and the epitope. According to this definition, an antibody is said to “specifically bind” to an epitope when it binds to that epitope via its antigen-binding domain more readily than it would bind to a random, unrelated epitope.

As used herein the terms “treat,” “treatment,” or “treatment of” (e.g., in the phrase “treating a patient with rheumatoid arthritis”) refers to reducing the potential for RA pathology, reducing the occurrence of RA symptoms, e.g., to an extent that the subject experiences reduced discomfort and debilitation. For example, treating can refer to the ability of a therapy when administered to a subject, to alleviate RA-mediated disease symptoms, signs, or causes. Treating also refers to mitigating or decreasing at least one clinical symptom and/or inhibition or delay in the progression of the condition and/or prevention or delay of the onset of a disease or illness.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

Mavrilimumab (see PCT Publication No. WO2007/110631 and PCT Publication No. WO2013/053767, both incorporated herein by reference in their entireties) is a human IgG4 monoclonal antibody designed to modulate macrophage activation, differentiation, and survival by targeting the GM-CSFRα. GM-CSFRα is the alpha chain of the receptor for granulocyte macrophage colony stimulating factor. Unless otherwise indicated by context, references herein to GM-CSF refer to human or non-human primate (e.g. cynomolgus) GM-CSF, normally human. GM-CSF normally binds to the extracellular domain of the mature GM-CSF receptor alpha chain. This binding is inhibited by mavrilimumab both in vitro and in vivo.

### Formulation and Administration

Mavrilimumab can be administered alone or in combination with other treatments, either simultaneously or sequentially dependent upon the condition to be treated. Mavrilimumab can be provided in combination with or in addition to one or more of the following: NSAIDs (e.g., cox inhibitors such as Ciclofenac or Celecoxib and other similar cox2 inhibitors), corticosteroids (e.g. prednisone oral and/or parenteral) and DMARDs e.g. Humira® (adalimumab), methotrexate, Arava, Enbrel® (Etanercept), Remicade® (Infliximab), Kineret (Anakinra), Rituxan® (Rituximab), Orencia® (abatacept), gold salts, antimalarials e.g. antimalarials (e.g., chloroquine, hydroxychloroquine), sulfasalazine, d-penicillamine, cyclosporin A, cyclophosphamide, azathioprine, leflunomide, certolizumab pegol (Cimzia®), toclizumab (Actmera®) and golimumab (Simponi®).

Mavrilimumab can be administered to individual rheumatoid arthritis (RA) patients, or to a population of RA patients.

The mavrilimumab dosage can be any dose from 30 mg per single dose up to at least 150 mg per single dose, e.g., 30 mg per single dose, 100 mg per single dose, or 150 mg per single dose. Single doses can be formulated for subcutaneous administration in a volume of 1 ml. Treatment can be administered at intervals of 14 days for 8 weeks, 10 weeks, 12 weeks, 14 weeks, 18 weeks, 20 weeks, 22 weeks, 24 weeks or more. Treatment can be continued in order to maintain or further improve clinical benefit.

Patients treated with mavrilimumab can continue to benefit from effects of the treatment for a sustained period after administration of the inhibitor, including clinical benefits such as a reduced DAS28-CRP. Clinical benefit can be maintained at the same level, or in some cases at a lower but still significant level of benefit, for a period of at least one month, at least two months, or at least three months following administration of mavrilimumab, for example following administration of at least three regular doses of mavrilimumab. Thus, in some embodiments, the methods of treating RA as provided herein can accommodate one or more pauses in treatment, while continuing to provide a therapeutic benefit to the patient for at least one month, at least two months, or at least three months.

Mavrilimumab can be administered in the form of a pharmaceutical composition, which can comprise at least one component in addition to the monoclonal antibody. Such pharmaceutical compositions can comprise, in addition to active ingredient, a pharmaceutically acceptable excipient, carrier, buffer, stabilizer or other materials well known to those skilled in the art. The precise nature of the carrier or other material will depend on the route of administration, which can be oral, intravenous, or by injection, e.g. subcutaneous injection. Liquid pharmaceutical compositions can comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included. For intravenous injection, or injection at the site of affliction, the active ingredient can be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included. Formulations can include excipients, or combinations of excipients, for example: sugars, amino acids and surfactants. Liquid formulations can include a wide range of antibody concentrations and pH. Formulations of mavrilimumab will depend upon the intended route of delivery: for example, formulations for pulmonary delivery can consist of particles with physical properties that ensure penetration into the deep lung upon inhalation; topical formulations can include viscosity modifying agents, which prolong the time that the drug is resident at the site of action. In certain embodiments, mavrilimumab can be prepared with a carrier that will protect it against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known to those skilled in the art. See, e.g., Robinson, J. R. ed., Sustained and Controlled Release Drug Delivery Systems, Marcel Dekker, Inc., New York, 1978.

### DAS28-CRP

Clinical benefit can be determined based on reduction in the Disease Activity Score 28 based on C-reactive protein (DAS28-CRP), for example decreasing DAS28-CRP by more than 1.2 from baseline, and/or reducing DAS28-CRP to less than 2.6.

DAS28-CRP can be determined as described previously (Smolen et al. Rheumatology 42:244-257 2003; Wells G, et al. Annals of the Rheumatic Diseases 68: 954-960 2009). As described by Wells et al., the DAS28 considers 28 tender and swollen joint counts, general health (GH; patient assessment of disease activity using a 100 mm visual analogue scale (VAS) with0=best, 100=worst), plus levels of an acute phase reactant (either erythrocyte sedimentation rate (ESR) (mm/h) or C-reactive protein (CRP) (mg/liter)). DAS28 values are calculated as follows:

DAS28-CRP=0.56*√(TJC28)+0.28*√(SJC28)+0.014*GH+0.36*1n(CRP+1)+0.96;
where CRP=C-reactive protein (mg/liter), GH=patient assessment of general health, TJC=tender joint count, and SJC=swollen joint count.

### ACR Criteria

Clinical benefit can be determined based on the ACR criteria or American College of Rheumatology Criteria (Arnett et al. Arthritis Rheum. 31 (3):315-324 1988). These standard criteria can be used in clinical trials to compare the effectiveness of various arthritis medications or treatments. ACR criteria is indicated as ACR20, ACR50, and ACR70. The RA patient can be scored at for example, ACR 20 (20 percent improvement) compared with no treatment (e.g baseline before treatment) or treatment with placebo. Typically it is convenient to measure improvement compared with the patient's baseline value. The ACR 20 criteria can include 20% improvement in both tender (painful) joint count and swollen joint count plus a 20% improvement in at least 3 of 5 additional measures:
1. patient's pain assessment by visual analog scale (VAS),
2. patient's global assessment of disease activity (VAS),
3. physician's global assessment of disease activity (VAS),
4. patient's self-assessed disability measured by the Health Assessment Questionnaire (HAQ), and
5. acute phase reactants, CRP or ESR.

The ACR 50 and 70 are defined analogously. Clinical trials report the percentage of study participants who achieve ACR20, ACR50, and ACR70. For example, if a study reported that 55 percent of patients achieved ACR20, that means 55 percent of patients in the study achieved a 20 percent improvement in tender or swollen joint counts as well as 20 percent improvement in three of the other five criteria. If a clinical trial reports that 40 percent of patients achieved ACR50, that means 40 percent of patients in the study achieved a 50 percent improvement in tender or swollen joint counts as well as 50 percent improvement in three of the other five criteria.

The HAQ, introduced in 1980, was among the first patient-reported outcome instruments designed to represent a model of patient-oriented outcome assessment (Bruce & Fries, Clin. Exp. Rheumatol. 23(suppl. 39): S14-S18 2005).

### Health Assessment Questionnaire Disability Index (HAQ-DI)

The HAQ-DI is a standardized measure of a patient's reported disability, determined the patient's reporting of his or her ability to perform everyday activities. Detailed information on the HAQ and the HAQ-DI has been published (Bruce & Fries, Clin. Exp. Rheumatol. 23(suppl. 39): S14-S18 2005).

### Demin Longitudinal Meta-Analysis

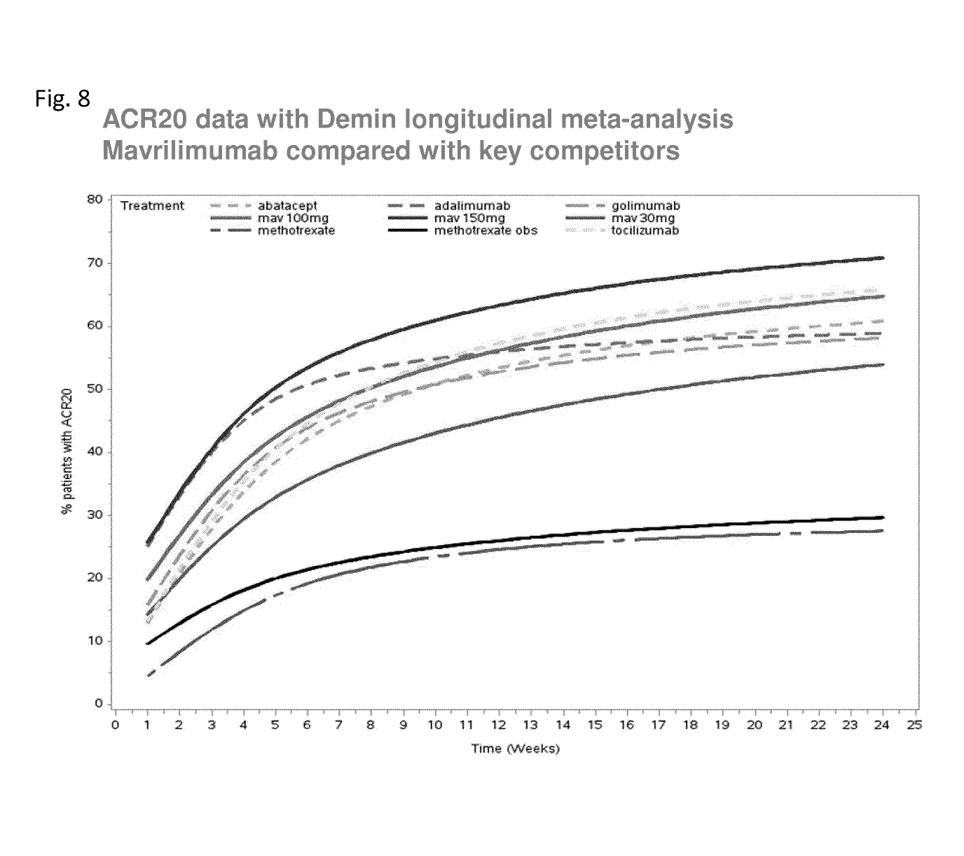

Demin Longitudinal Meta-analysis was used for an exploratory cross-trial comparison of the longitudinal ACR20 profile observed in the present clinical study compared to other biologics in a similar population. The Demin analysis fitted an Emax model to the ACR20 profile over time for all the approved biologics for rheumatoid arthritis. We used the model parameters in the Demin publication (Demin, I., et al., Clin. Pharmacol. Therap. 92:352-359) to recreate the profiles and then fitted the same Emax model to the current study data and overlaid the results. The results provided below and in FIG. 8 show the ACR20 results observed in the current study were highly competitive in terms for speed of onset as well as the maximum ACR20 response rate.

### SF-36®

The SF-36® Health Survey that asks 36 questions to measure functional health and well-being from the patients point of view. It can be used across age (18 and older), disease, and treatment group. It is not a disease-specific health survey.

### CDAI

Clinical Disease Activity Index (CDAI) is a useful clinical composite score for following patients with rheumatoid arthritis. CDAI is calculated as: SJC(28)+TJC(28)+PGA+EGA. Where SJC(28) is the Swollen 28-Joint Count (shoulders, elbows, wrists, MCPs, PIPs including thumb IP, knees); TJC(28) is the Tender 28-Joint Count (shoulders, elbows, wrists, MCPs, PIPs including thumb IP, knees); PGA is the Patient Global disease Activity (patient's self-assessment of overall RA disease activity on a scale of 1 to 10, where 10 is maximal activity); and EGA is the Evaluator's Global disease Activity (evaluator's assessment of overall RA disease activity on a scale of 1 to 10, where 10 is maximal activity). A CDAI≦2.8 is interpreted as remission; a CDAI>2.8 and ≦10 is interpreted as Low Disease Activity; a CDAI>10 and ≦22 is interpreted as Moderate Disease Activity; and a CDAI of >22 is interpreted as High Disease Activity.

The following disclosed embodiments are merely representative. Thus, specific structural, functional, and procedural details disclosed in the following examples are not to be interpreted as limiting.

### EXAMPLES

### Example 1

### Study Design Overview

A total of 420 subjects were screened, with 326 subjects subsequently being randomized into the four cohorts. Of these, 284 subjects were included in the ITT population. All cohorts were well balanced in terms of baseline and disease characteristics. A Phase 2b randomized, double blind, placebo controlled, multiple ascending dose study was performed to evaluate the efficacy, safety and tolerability of mavrilimumab in subjects with RA. The trial permitted evaluation of a number of factors including clinical outcomes in RA, the relationship between dosage and safety and efficacy, and the pharmacokinetics and immunogenicity of mavrilimumab. Cohorts were treated with 30 mg, 100 mg, and 150 mg doses.

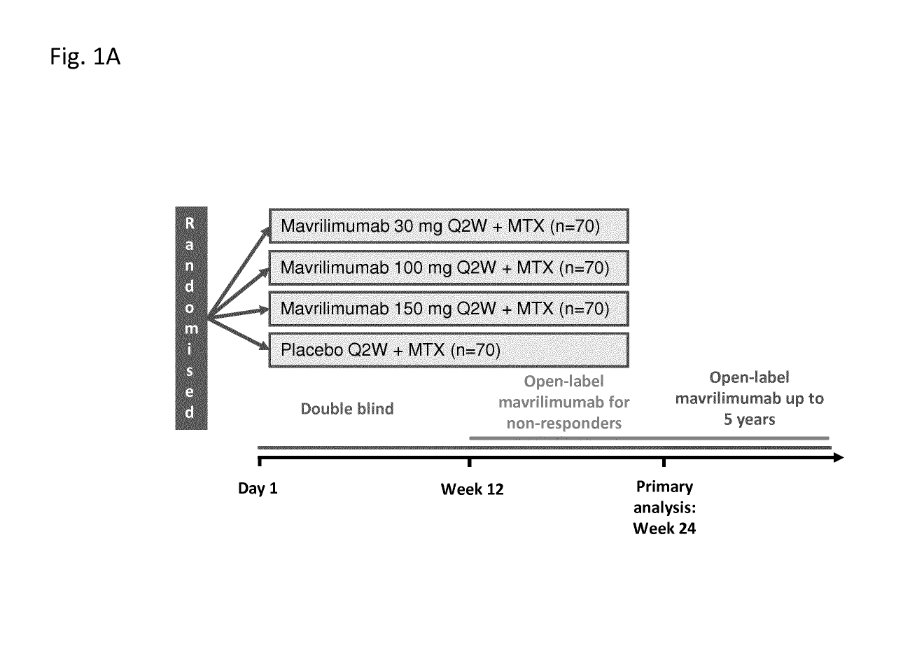

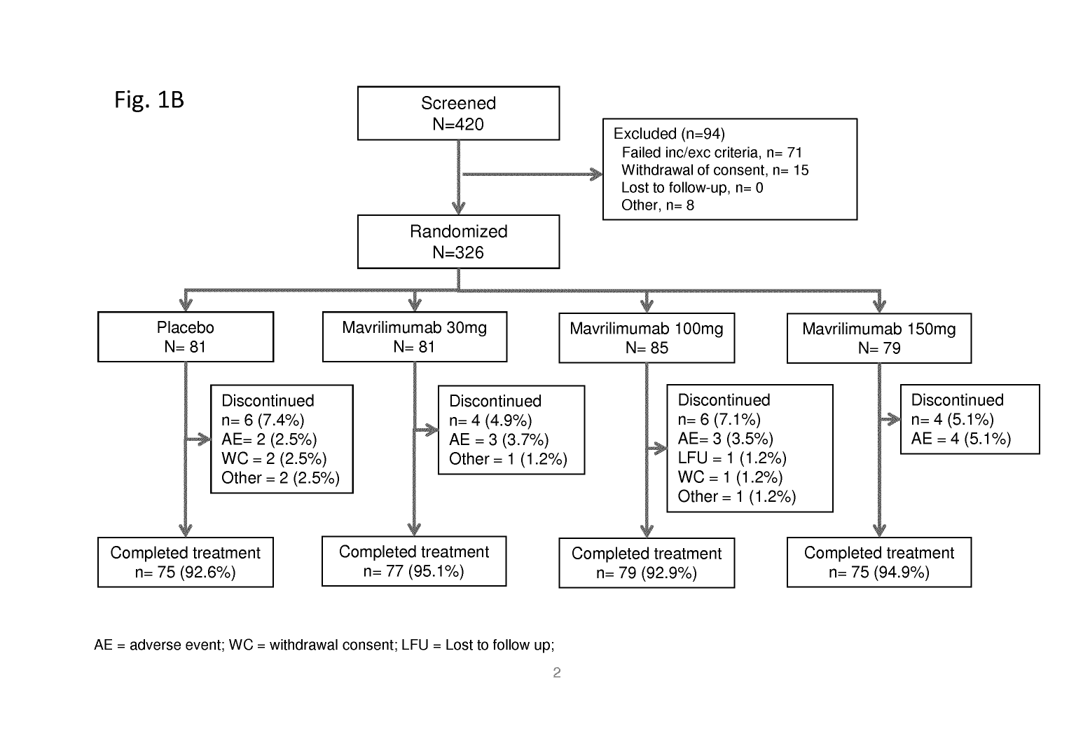

The primary objective of the clinical trial study was to evaluate the efficacy of three subcutaneous (SC) doses of mavrilimumab (i.e., 30 mg, 100 mg, and 150 mg) compared with placebo in combination with MTX in subjects with inadequate responder to non-biologic DMARDs. FIGS. 1A and 1B show the study design. A first primary endpoint was the proportion of combined mavrilimumab-treated subjects achieving an improvement of 1.2 from baseline in DAS28-CRP versus placebo at Week 12 (Day 85). The response rate was calculated, where a responder was defined as a subject showing a decrease of more than 1.2 from their baseline DAS28-CRP. A second primary end point was the ACR20 at Week 24 (Day 169). Secondary endpoints included ACR 20, ACR 50 and ACR 70 responses, remission rate (DAS28-CRP<2.6) and safety and tolerability of mavrilimumab. Additional assessments included the time to onset of remission, an improvement of 1.2 points from baseline, swollen and tender joint count and measurements of acute phase reactants (CRP and ESR). Patient reported outcomes including the Health Assessment Questionnaire Disability Index (HAQ-DI) (Fries et al. Arthritis and Rheumatism 23: 137-145 1980) were also measured.

Inclusion criteria comprised a diagnosis of adult onset RA defined by the 2010 ACR/EULAR classification, at least moderately active disease as defined by DAS28 (CRP)≧3.2 at Screening and DAS28 (ESR)≧3.2 at Day 1, at least 4 swollen joints at Screening and Day 1, and subjects with inadequate response to one or more conventional DMARDs. Exclusion criteria included previous treatment with biologic DMARDs discontinued for lack of efficacy, however, previous use of one biologic DMARD discontinued for other reasons than lack of efficacy was allowed.

### Statistical Methods

Sample size calculations were based on the primary efficacy endpoint (change of 1.2 points in DAS28-CRP at Week 12). A placebo response rate of 10%, a 15% drop-out rate, a two-sided Type 1 error of 0.05, and a 2:1 (active:placebo) randomization ratio were assumed, providing a total sample size of 216 subjects with 86% power to detect a 20% difference in response rates for an analysis based on a two-sided Fisher's exact test.

All response rates, including the primary endpoint, ACR20, ACR50 and ACR70, were analyzed using Fisher's exact test. Changes from baseline in DAS28 score were analyzed using a mixed-model repeated measures analysis with a covariate for baseline DAS28. The DAS28 European League Against Rheumatism (EULAR) response criteria were analyzed using a Cochran-Mantel-Haenszel test. Improvement in DAS28 was categorised using the EULAR response criteria as shown below in Table 1AA:

TABLE 1AA
Improvement in DAS28 Score
DAS28 Improvement

| DAS score | | | |
|---|---|---|---|
| at visit | >1.2 | 0.6-1.2 | <0.6 |
| <3.2 | Good Response | Moderate response | No Response |
| 3.2-5.1 | Moderate response | Moderate response | No Response |
| >5.1 | Moderate response | No Response | No Response |

Time-to-onset of response was analysed using a non-parametric log-rank test.

All efficacy analyses were conducted using data from the intent-to-treat (ITT) population. Sensitivity analyses were conducted using the per protocol (PP) population. Each analysis was conducted to compare the combined placebo and combined mavrilimumab groups, followed by comparison of the combined placebo group with each of the mavrilimumab dose cohorts. Analysis of safety data was carried out on the safety population, defined as all subjects who received any dose of study medication.

For the primary endpoint as well as the other responder analyses, a non-responder imputation was used for subjects who withdrew from study treatment, changed the dose of background methotrexate or received other RA medication. Other missing data points were imputed using last-observation-carried-forward methodology. No imputation was applied for the DAS28 change from baseline analysis.

Baseline Characteristics of the mITT Population

As shown in Table 1AB, participating patients were located in numerous countries in Europe, South America, and Africa. Twenty-five percent of the patients were from South America.

TABLE 1AB
Countries

| | | Placebo | | Mav 30 mg | | Mav 100 mg | | Mav 150 mg | |
|---|---|---|---|---|---|---|---|---|---|
| Region | Country | (N = 81) | | (N = 81) | | (N = 85) | | (N = 79) | |
| Europe | Bulgaria | 3 | (3.7%) | 2 | (2.5%) | 2 | (2.4%) | 2 | (2.5%) |
| | Czech Republic | 13 | (16.0%) | 13 | (16.0%) | 14 | (16.5%) | 13 | (16.5%) |
| | Estonia | 6 | (7.4%) | 6 | (7.4%) | 6 | (7.1%) | 5 | (6.3%) |
| | Germany | 2 | (2.5%) | 2 | (2.5%) | 3 | (3.5%) | 2 | (2.5%) |
| | Hungary | 2 | (2.5%) | 1 | (1.2%) | 2 | (2.4%) | 2 | (2.5%) |
| | Poland | 9 | (11.1%) | 8 | (9.9%) | 10 | (11.8%) | 10 | (12.7%) |
| | Russia | 8 | (9.9%) | 9 | (11.1%) | 8 | (9.4%) | 8 | (10.1%) |
| | Serbia | 6 | (7.4%) | 6 | (7.4%) | 6 | (7.1%) | 6 | (7.6%) |
| | Spain | 0 | | 0 | | 1 | (1.2%) | 0 | |
| | Ukraine | 10 | (12.3%) | 11 | (13.6%) | 12 | (14.1%) | 10 | (12.7%) |
| South America | Argentina | 7 | (8.6%) | 8 | (9.9%) | 8 | (9.4%) | 8 | (10.1%) |
| | Chile | 10 | (12.3%) | 10 | (12.3%) | 9 | (10.6%) | 8 | (10.1%) |
| | Colombia | 5 | (6.2%) | 4 | (4.9%) | 4 | (4.7%) | 4 | (5.1%) |
| Africa | South Africa | 0 | | 1 | (1.2%) | 0 | | 1 | (1.3%) |

Table 1B shows the mean age in years, percentage of male patients to female patients, mean weight and mean Body Mass Index (BMI) of the participating patients.

TABLE 1B
Demographics

| | | | Mav | Mav | Mav |
|---|---|---|---|---|---|
| | | Placebo | 30mg | 100 mg | 150 mg |
| Characteristic | | (N = 81) | (N = 81) | (N = 85) | (N = 79) |
| Age (years) | Mean | 52.8 | 51.2 | 50.8 | 52.6 |
| Female | % | 92.6 | 86.4 | 82.4 | 84.8 |
| Male | % | 7.4 | 13.6 | 17.6 | 15.2 |
| Weight (Kg) | Mean | 73.0 | 72.5 | 71.8 | 75.9 |
| BMI (Kg/m²) | Mean | 27.5 | 27.3 | 26.3 | 28.4 |

Table 1C shows the rheumatoid arthritis history of the patients in the four cohorts, including the mean dose of methotrexate (MTX) patients were receiving.

TABLE 1C
RA History

| | | | Mav | Mav | Mav |
|---|---|---|---|---|---|
| | | Placebo | 30 mg | 100 mg | 150 mg |
| | | (N = 81) | (N = 81) | (N = 85) | (N = 79) |
| Disease | Median | 7.1 | 7.4 | 6.4 | 7.3 |
| duration | | | | | |
| (years) | | | | | |
| MTX dose | Mean | 15.0 | 14.6 | 15.1 | 14.6 |
| (mg/week) | | | | | |
| Concomitant | % | 53.1 | 61.7 | 60.0 | 58.2 |
| steroids | | | | | |
| Biologic | % | 14.8 | 14.8 | 15.3 | 12.7 |
| exposed | | | | | |
| RF or ACPA | % | 80.2 | 86.4 | 81.2 | 79.7 |
| +ve | | | | | |

Table 1D shows the mean baseline disease activity of the patients in the four cohorts to which improvements were determined.

TABLE 1D
Baseline disease activity ACR components and DAS28 score

| | | Placebo | | Mav 30 mg | | Mav 100 mg | | Mav 150 mg | |
|---|---|---|---|---|---|---|---|---|---|
| | | (N = 81) | | (N = 81) | | (N = 85) | | (N = 79) | |
| DAS28 CRP | Mean (SD) | 5.8 | (0.8) | 5.7 | (0.9) | 5.9 | (0.9) | 5.7 | (0.8) |
| DAS28 ESR | Mean (SD) | 6.6 | (0.9) | 6.7 | (1.0) | 6.7 | (0.9) | 6.5 | (0.9) |
| Swollen joint | Mean (SD) | 14.4 | (6.9) | 17.8 | (10.1) | 16.8 | (8.6) | 15.7 | (7.1) |
| cnt | | | | | | | | | |
| Tender joint | Mean (SD) | 26.3 | (11.3) | 27.5 | (14.0) | 27.0 | (14.2) | 26.7 | (11.4) |
| cnt | | | | | | | | | |
| Patient | Mean (SD) | 65 | (17) | 64 | (19) | 64 | (18) | 62 | (19) |
| global (mm) | | | | | | | | | |
| Patient | Mean (SD) | 62 | (19) | 63 | (19) | 64 | (19) | 62 | (20) |
| pain (mm) | | | | | | | | | |
| Physician | Mean (SD) | 6.6 | (1.5) | 6.6 | (1.5) | 6.8 | (1.3) | 6.4 | (1.5) |
| global (cm) | | | | | | | | | |
| HAQ-DI | Mean (SD) | 1.63 | (0.48) | 1.52 | (0.62) | 1.58 | (0.52) | 1.58 | (0.53) |
| CRP mg/L | Geometric | 6.1 | (145%) | 4.7 | (161%) | 7.6 | (107%) | 5.2 | (118%) |
| | mean | | | | | | | | |
| | (CV %) | | | | | | | | |
| ESR mm/hr | Geometric | 36.2 | (51%) | 39.3 | (52.6%) | 38.1 | (52.8%) | 35.4 | (52.9%) |
| | mean | | | | | | | | |
| | (CV %) | | | | | | | | |

Three hundred twenty six patients participated in the study and were distributed as shown in FIGS. 1A and 1B. Over 90% of subjects completed treatment. Approximately 50% of placebo subjects withdrew before Day 169, mostly due to lack of efficacy. There was an option to enter the open label extension if not responding. The demographics were generally well balanced but there was a higher proportion of male subjects on active than placebo and a higher proportion of subjects on steroids on active than placebo. Approximately 20% of subjects were sero-negative. The baseline DAS28 disease activity was typical of RA population.

### Example 2

### Efficacy

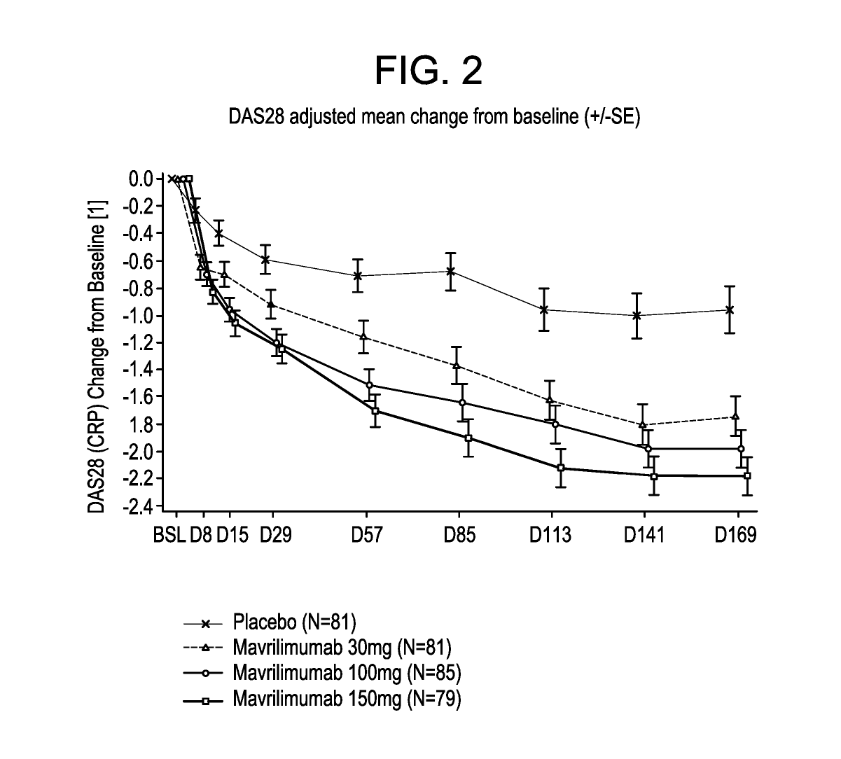

Table 2A shows the co-primary endpoint of DAS28 change from baseline after 12 weeks (Day 85). The mean baseline DAS28 activity of the four cohorts ranged from 5.7 to 5.9. After Week 12, the 150 mg treated cohort saw a −1.90 adjusted mean change from baseline compared to on a −0.68 adjusted mean change from baseline for the placebo group. This represented a −1.22 improvement of the 150 mg treated patients over those receiving the placebo. FIG. 2 shows the DAS28 adjusted mean change from baseline from the beginning of treatment to 85 days and then extending out to at least 169 days for treated patients. At 169 days, the 150 mg treated cohort saw an adjusted mean change from baseline of almost −2.2 compared to the placebo group which saw only around a −1.1 adjusted mean change from baseline just short of 160 days.

TABLE 2A
Co-Primary Endpoint: DAS28 (CRP) change from baseline: Day 85

| | Placebo | Mav 30 mg | Mav 100 mg | Mav 150 mg |
|---|---|---|---|---|
| Endpoint | (N = 81) | (N = 81) | (N = 85) | (N = 79) |
| DAS28 | 5.8 | 5.7 | 5.9 | 5.7 |
| Baseline | | | | |
| DAS28 (CRP) | −0.68 (0.14) | −1.37 (0.14) | −1.64 (0.13) | −1.90 (0.14) |
| Adj mean | | | | |
| Change (SE) | | | | |

| | | | |
|---|---|---|---|
| Difference from placebo | −0.69 | −0.96 | −1.22 |
| (95% CI) | (−1.06, −0.31) | (−1.33, −0.58) | (−1.60, −0.84) |
| P-value | <0.001 | <0.001 | <0.001 |

One secondary endpoint was Low Disease Activity (DAS28 score 3.2 -2.6). As shown in Table 2B, Low Disease Activity was achieved by 42% patients receiving 150 mg versus only 9% on placebo at day 169.

TABLE 2B
Low Disease Activity (DAS28 score 3.2-2.6)

| | | Number (%) | | Difference in % | 95% | |
|---|---|---|---|---|---|---|
| Treatment | | Responders | | (Mavrilimumab - | Confidence | |
| Group | N | (Day 169) | | Placebo) | Interval | P-Value |
| Placebo | 81 | 7 | (8.6%) | | | |
| Mavrilimumab | 81 | 27 | (33.3%) | 24.7% | 12.7, 36.6 | <0.001 |
| 30 mg | | | | | | |
| Mavrilimumab | 85 | 27 | (31.8%) | 23.1% | 11.5, 34.8 | <0.001 |
| 100 mg | | | | | | |
| Mavrilimumab | 79 | 33 | (41.8%) | 33.1% | 20.7, 45.6 | <0.001 |
| 150 mg | | | | | | |

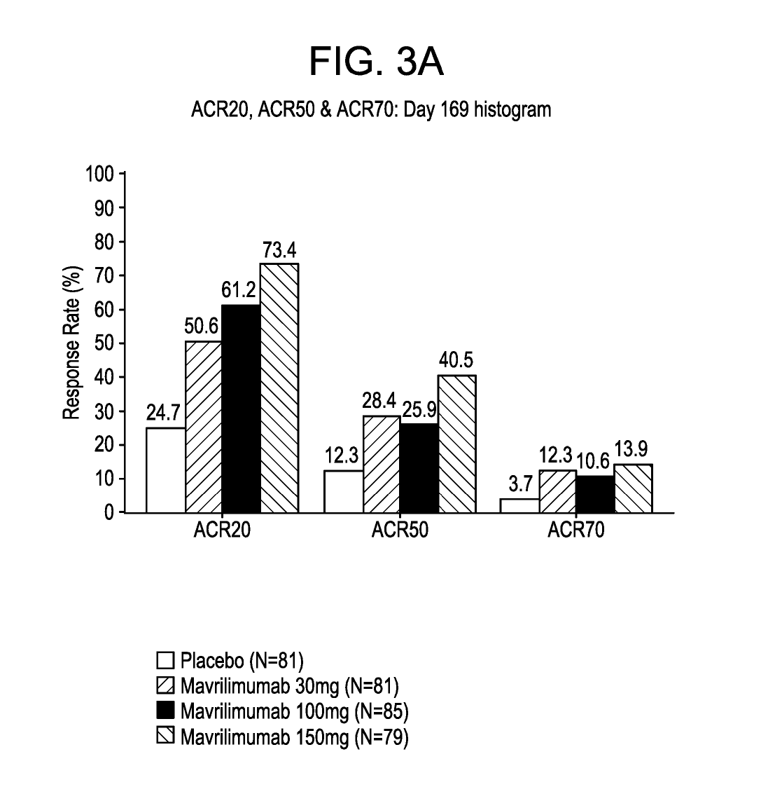

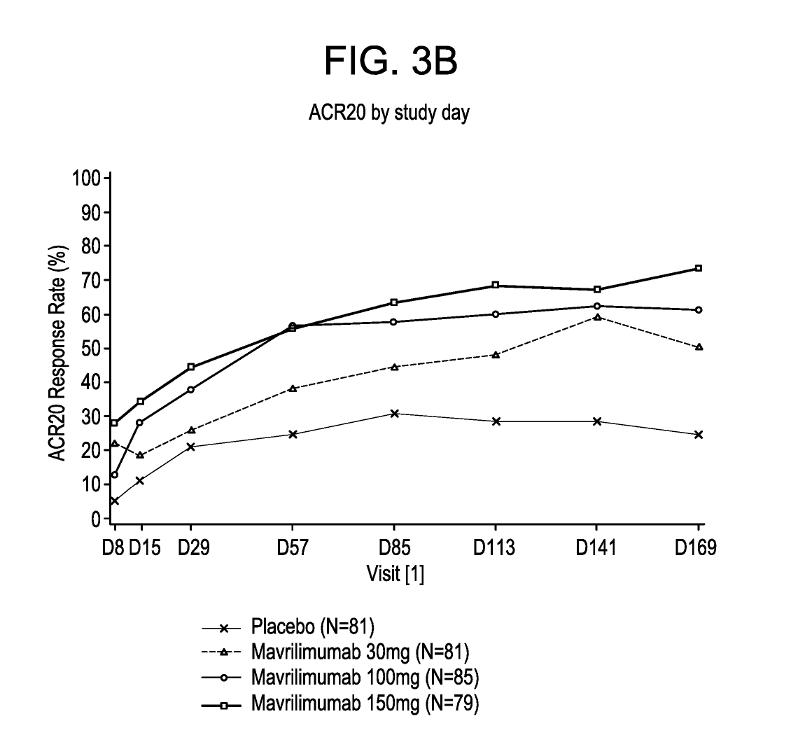

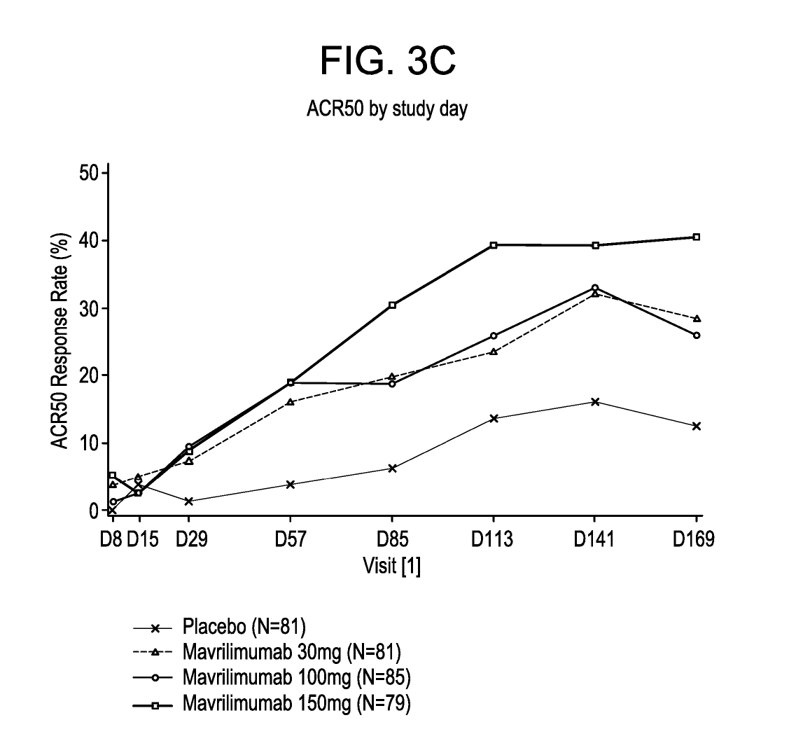

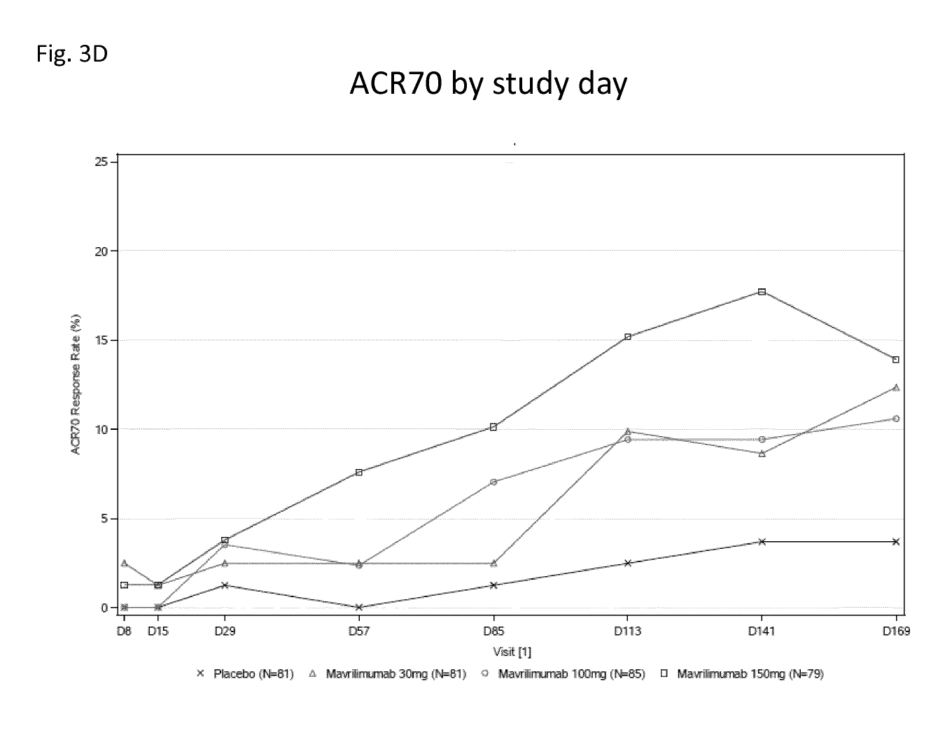

Table 3A shows the co-primary endpoint of ACR efficacy responses after 24 weeks (Day 169). After 24 weeks, 73.4% of those receiving 150 mg doses of mavrilimumab achieved ACR20 versus 24.7% for placebo. After 24 weeks, 40.5% of those receiving 150 mg doses of mavrilimumab achieved ACR20 versus 12.3% for placebo. After 24 weeks, 13.9% of those receiving 150 mg doses of mavrilimumab achieved ACR20 versus 3.7% for placebo. The results shown in Table 3A are also shown in the histogram of FIG. 3A.

TABLE 3A
ACR Efficacy Responses (ACR20, ACR50, and ACR70) Day 169

| | Placebo | Mav 30 mg | Mav 100 mg | Mav 150 mg |
|---|---|---|---|---|
| Endpoint | (N = 81) | (N = 81) | (N = 85) | (N = 79) |
| ACR20 | 24.7% | 50.6% | 61.2% | 73.4% |
| Diff vs pbo | | 25.9 | 36.5 | 48.7 |
| p-value | | <0.001 | <0.001 | <0.001 |
| ACR50 | 12.3% | 28.4% | 25.9% | 40.5% |
| Diff vs pbo | | 16.0 | 13.5 | 28.2 |
| p-value | | 0.013 | 0.030 | <0.001 |
| ACR70 | 3.7% | 12.3% | 10.6% | 13.9% |
| Diff vs pbo | | 8.6 | 6.9 | 10.2 |
| p-value | | 0.079 | 0.133 | 0.026 |
| ACRn | 13.2% | 29.0% | 30.2% | 40.7% |
| Diff vs pbo | | 15.8% | 17% | 27.5 |
| p-value | | 0.009 | 0.004 | <0.001 |
| ACR hybrid | 0% | 20% | 46.6% | 50% |
| (Median) | | <0.001 | <0.001 | <0.001 |

FIGS. 3B, 3C, and 3D show day-by-day ACR20, ACR50, and ACR70 results, respectively. As can been, a higher percentage of the 150 mg mavrilimumab-treated patients consistently achieved the ACR20, ACR50, and ACR70 criteria as compared to placebo.

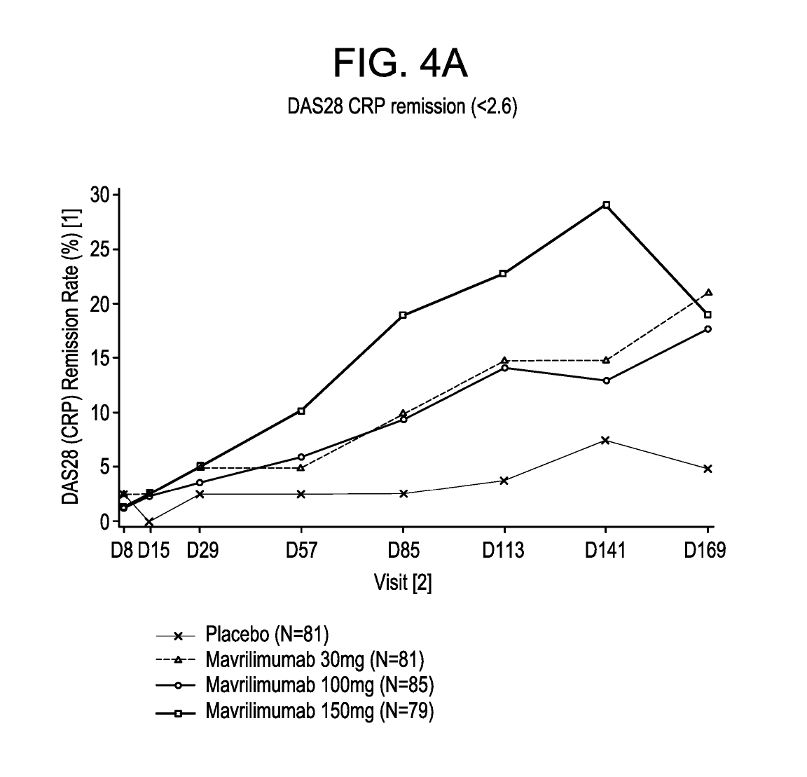

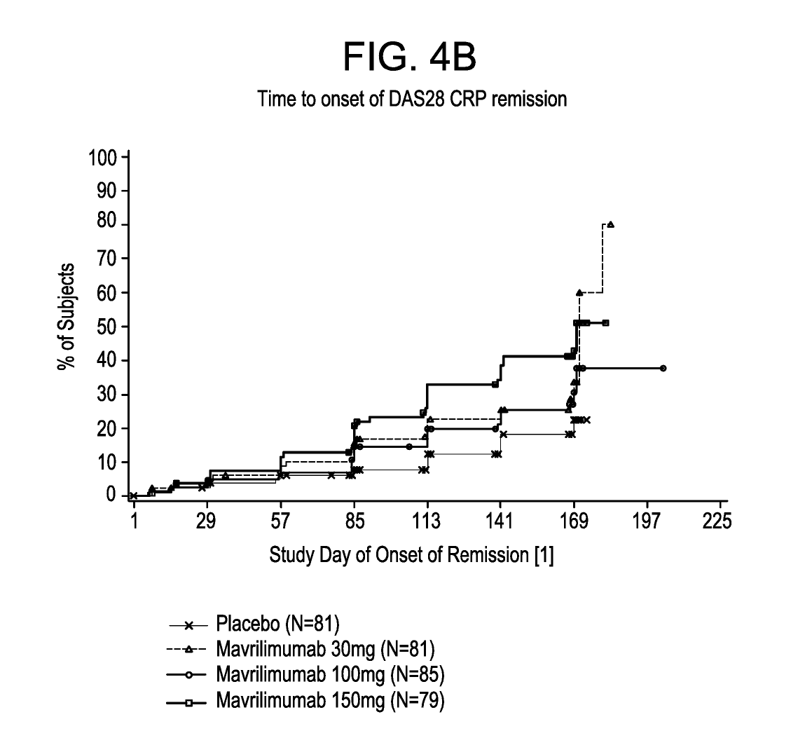

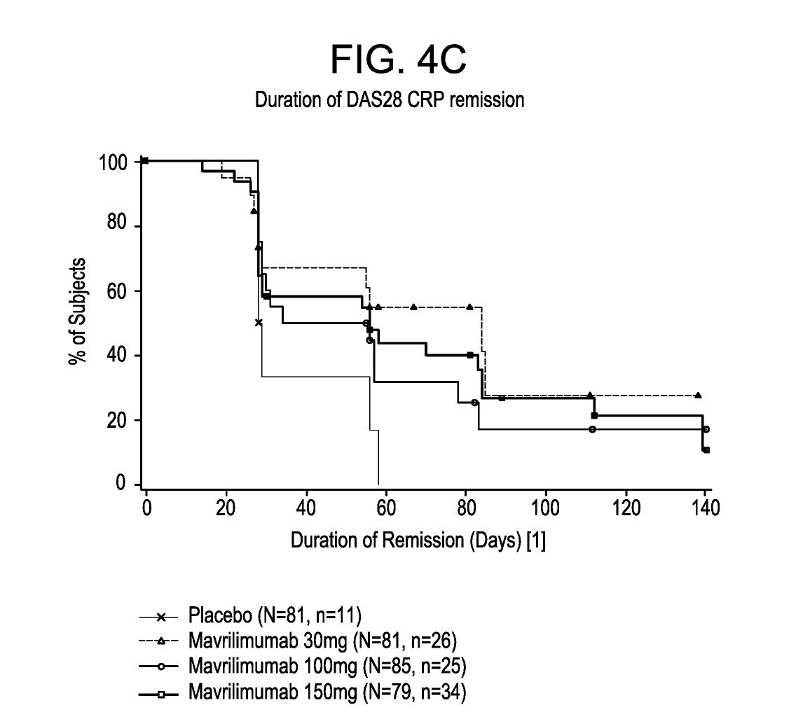

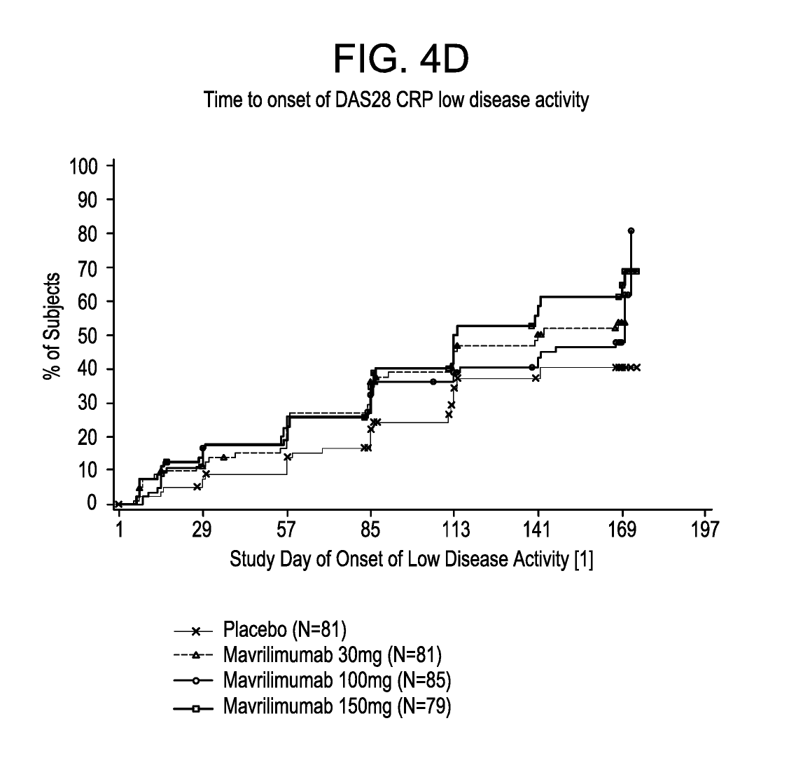

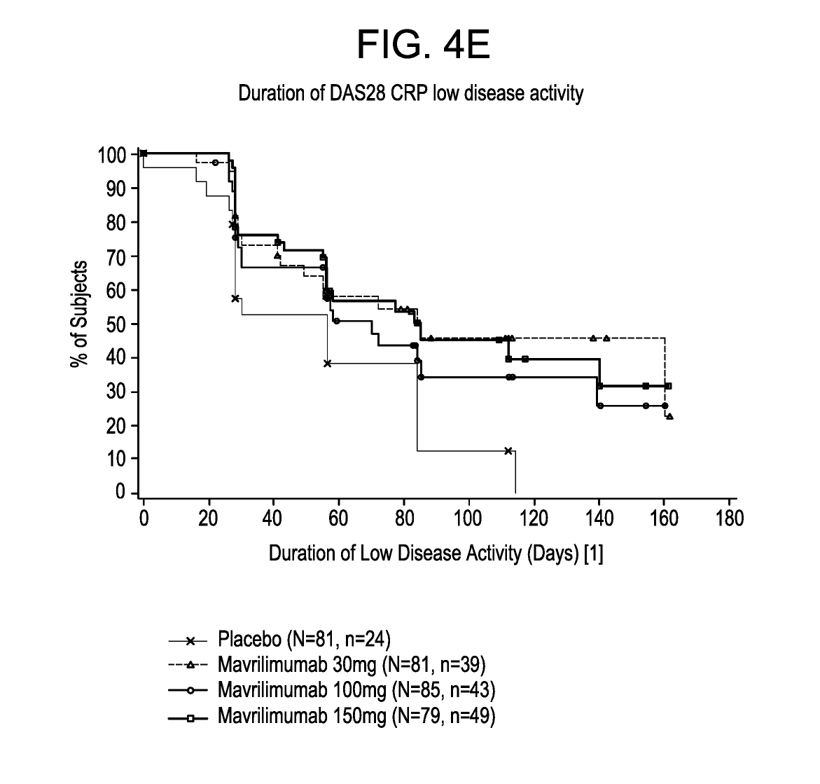

Table 4 shows the percentage of patients with a DAS28 score of <2.6 (remission) after 24 weeks (Day 169). After 24 weeks, 19% of the 150 mg mavrilimumab-treated patients were in remission. FIG. 4A is a graph showing from the beginning of treatment to Day 169 the rate of remission.

TABLE 4
Remission (DAS28 score <2.6)

| | | Mav | Mav | Mav |
|---|---|---|---|---|
| | Placebo | 30 mg | 100 mg | 150 mg |
| Day 169 | (N = 81) | (N = 81) | (N = 85) | (N = 79) |
| DAS28 CRP remission | 4.9% | 21.0% | 17.6% | 19.0% |

| | | | |
|---|---|---|---|
| Difference from placebo | 16.0 | 12.7 | 14.0 |
| (95% CI) | (6.0, 26.1) | (3.3, 22.1) | (4.2, 23.9) |
| p-value | 0.004 | 0.014 | 0.007 |

### Time to Response and Duration of Response

FIG. 4B shows that a dose response is observed in the time to onset of DAS28 CRP remission with the 150 mg dose decreasing the time as compared to placebo.

FIG. 4C shows a longer duration of DAS28-CRP remission for patients treated with 150 mg mavrilimumab than placebo.

FIG. 4D shows a dose response corresponding to faster time to onset of DAS28 CRP remission between 150 mg mavrilimumab-treated patients and placebo.

FIG. 4E shows a longer duration of DAS28-CRP low disease activity for patients treated with 150 mg mavrilimumab than placebo.

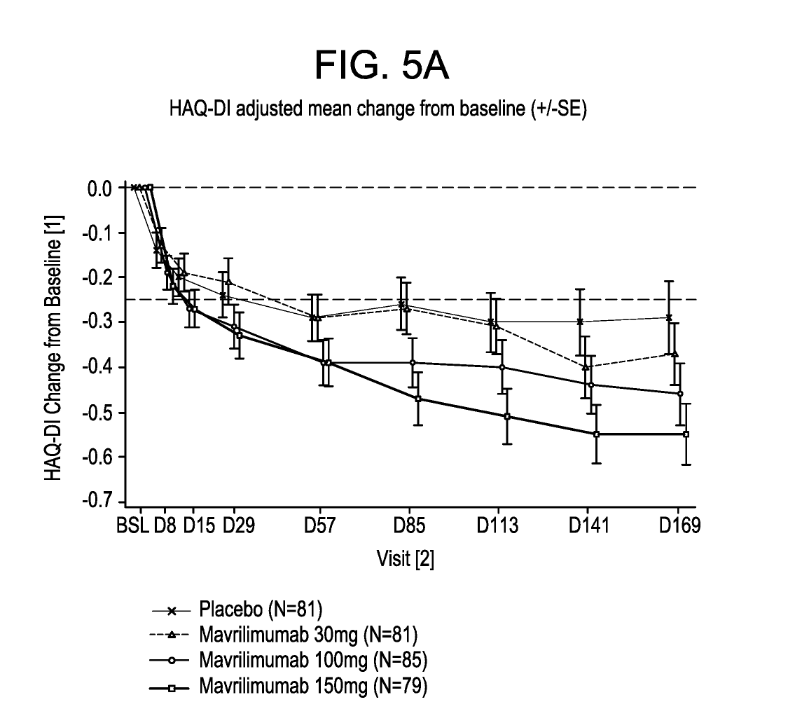

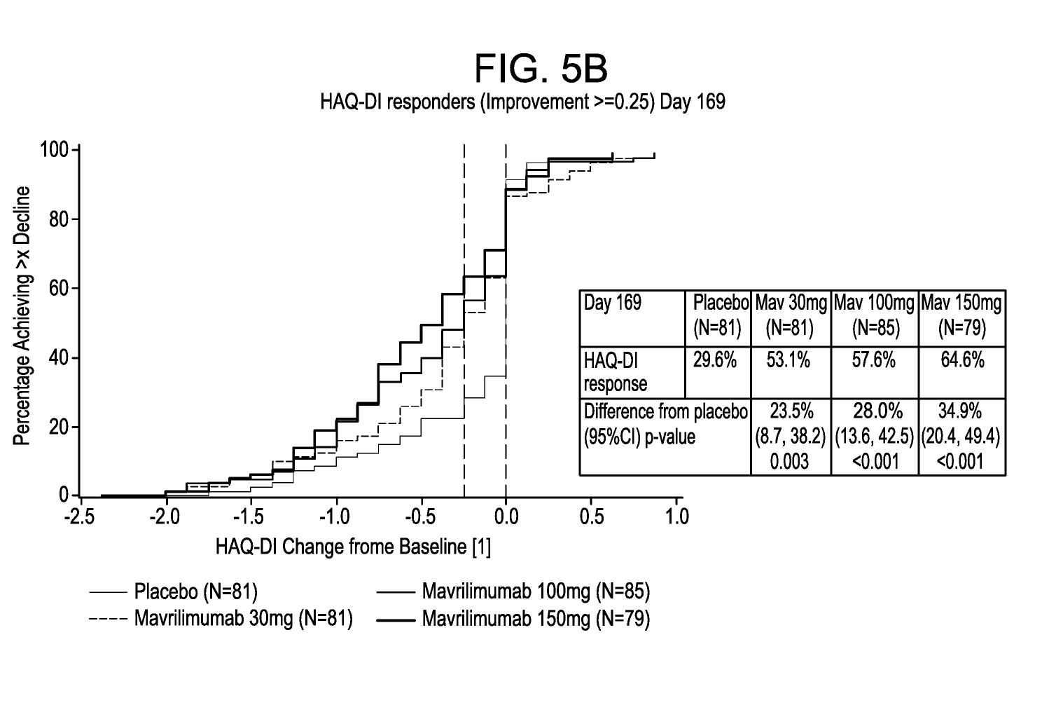

Table 5A shows that after 24 weeks (Day 169), 150 mg mavrilimumab-treated patients exhibited a −0.55 adjusted mean change as compared to −0.29 for placebo (i.e, a −0.26 difference from placebo). FIG. 5A shows the HAQ-DI adjusted mean change from baseline (+/−SE) of the four cohorts from beginning of treatment to Day 169.

TABLE 5A
HAQ-DI change from baseline at Day 169

| | | Mav | Mav | Mav |
|---|---|---|---|---|
| | Placebo | 30 mg | 100 mg | 150 mg |
| Endpoint | (N = 81) | (N = 81) | (N = 85) | (N = 79) |
| HAQ-DI Baseline | 1.63 | 1.52 | 1.58 | 1.58 |
| HAQ-DI | −0.29 | −0.37 | −0.46 | −0.55 |
| Adj mean Change (SE) | (0.08) | (0.07) | (0.07) | (0.07) |

| | | | |
|---|---|---|---|
| Difference from placebo | −0.08 | −0.16 | −0.26 |
| (95% CI) | (−0.29, | (−0.37, | (−0.47, |
| | 0.14) | 0.04) | −0.05) |
| P-value | 0.479 | 0.124 | 0.017 |

As shown in Table 5B, the 150 mg mavrilimumab-treated cohort did better in HAQ-DI response than the placebo group (64.6% mavrilimumab-treated versus 29.6% placebo). FIG. 5B is a chart illustrating the results shown in Table 5B.

TABLE 5B
HAQ-DI responders (Improvement >=0.25) at Day 169

| | | | | Mav |
|---|---|---|---|---|
| | Placebo | Mav 30 mg | Mav 100 mg | 150 mg |
| Day 169 | (N = 81) | (N = 81) | (N = 85) | (N = 79) |
| HAQ-DI | 29.6% | 53.1% | 57.6% | 64.6% |
| response | | | | |

| | | | |
|---|---|---|---|
| Difference from placebo | 23.5% | 28.0% | 34.9% |
| (95% CI) p-value | (8.7, 38.2) | (13.6, 42.5) | (20.4, 49.4) |
| | 0.003 | <0.001 | <0.001 |

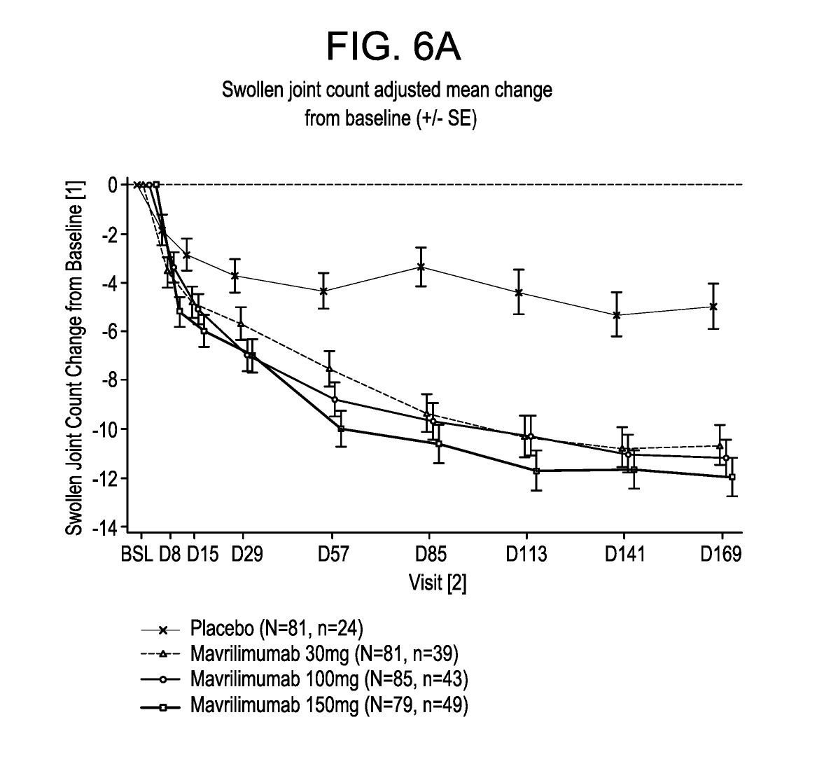

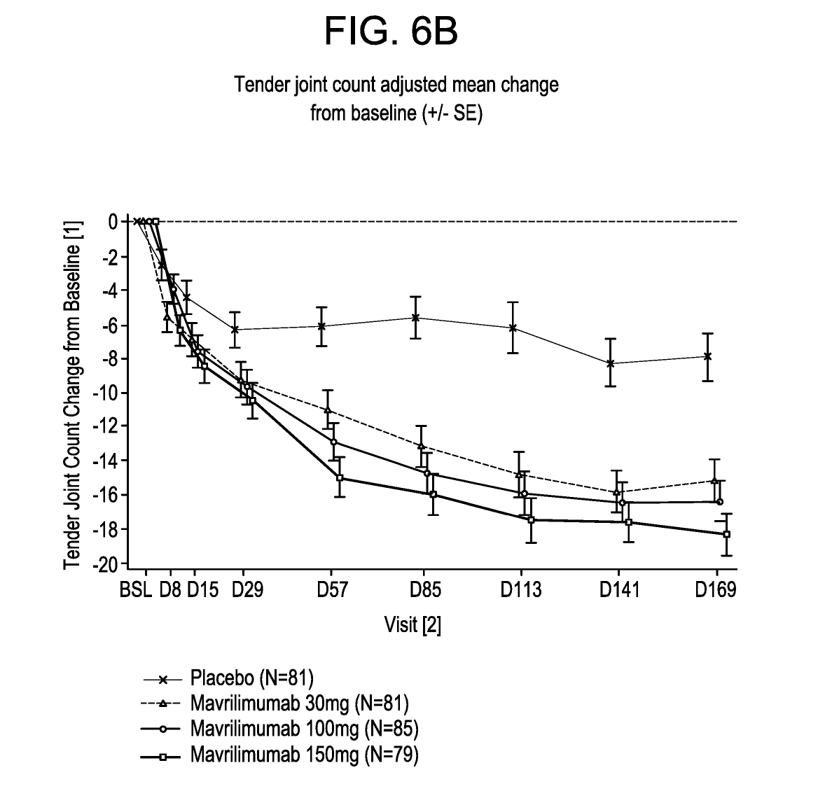

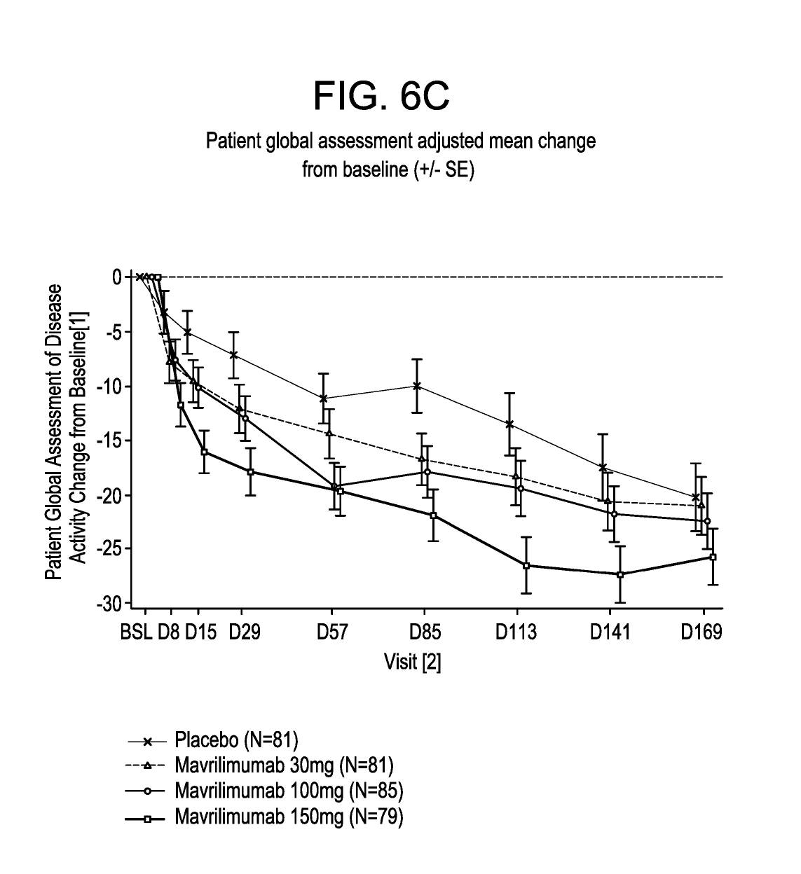

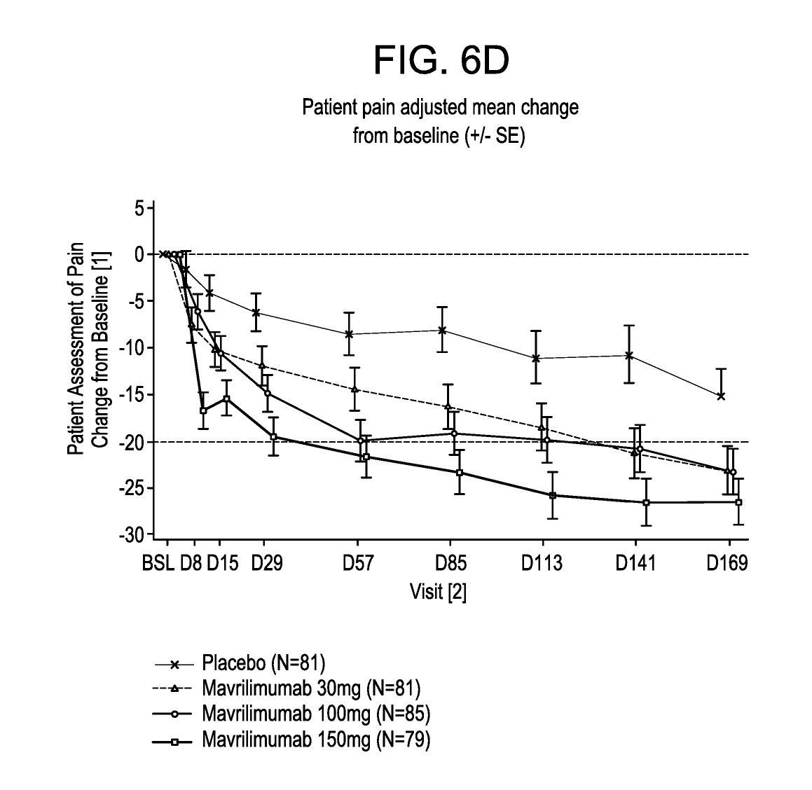

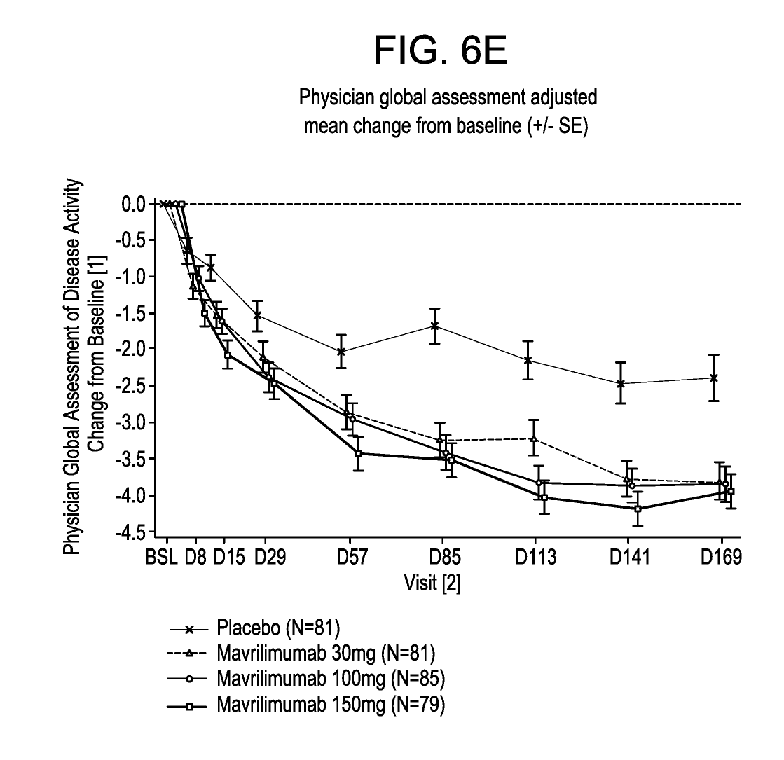

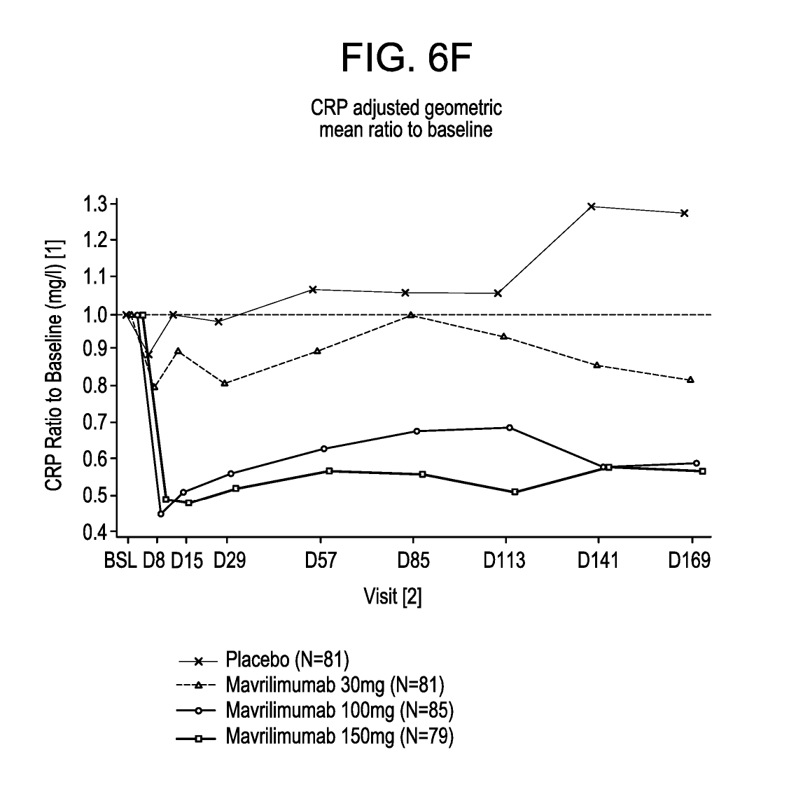

FIGS. 6A to 6F demonstrate additional, rapid patient benefit of 150 mg mavrilimumab-treatment, occurring as early as at one week and one dose and continuing for up to the 24 week time point. FIG. 6A shows swollen joint count adjusted mean change from baseline (+/−SE). FIG. 6B shows tender joint count adjusted mean change from baseline (+/−SE). FIG. 6C shows the Patient Global Assessment adjusted mean change from baseline (+/−SE). FIG. 6D shows Patient Pain adjusted mean change from baseline (+/−SE). FIG. 6E shows the Physician Global Assessment adjusted mean change from baseline (+/−SE). FIG. 6F shows the CRP adjusted geometric mean ratio to baseline.

Tables 7A and 7B show patient fatigue data for patients treated with 150 mg mavrilimumab.

TABLE 7A
Patient Fatigue score

| | Placebo | Mav 30 mg | Mav 100 mg | Mav 150 mg |
|---|---|---|---|---|
| Visit | (N = 81) | (N = 81) | (N = 85) | (N = 79) |
| Day 29 | 3.30 | 4.19 | 4.85 | 4.97 |
| Difference from placebo | | 0.89 | 1.56 | 1.67 |
| (95% CI) | | (−1.66, 3.43) | (−0.93, 4.04) | (−0.86, 4.21) |
| p-value | | 0.494 | 0.220 | 0.195 |
| Day 85 | 3.61 | 4.59 | 5.07 | 6.84 |
| Difference from placebo | | 0.98 | 1.46 | 3.23 |
| (95% CI) p-value | | (−1.72, 3.68) | (−1.20, 4.13) | (0.54, 5.92) |
| | | 0.476 | 0.280 | 0.019 |
| Day 141 | 5.85 | 6.19 | 6.72 | 8.04 |
| Difference from placebo | | 0.34 | 0.87 | 2.19 |
| (95% CI) | | (−2.84, 3.52) | (−2.26, 3.99) | (−0.95, 5.32) |
| p-value | | 0.834 | 0.586 | 0.171 |
| Day 169 | 4.53 | 5.72 | 6.80 | 8.45 |

| | | | |
|---|---|---|---|
| Difference from placebo | 1.18 | 2.27 | 3.92 |
| (95% CI) | (−1.99, 4.35) | (−0.83, 5.37) | (0.81, 7.02) |
| p-value | 0.463 | 0.151 | 0.014 |

TABLE 7B
FACIT fatigue response

| | | | | Mav |
|---|---|---|---|---|
| | Placebo | Mav 30 mg | Mav 100 mg | 150 mg |
| Day 169 | (N = 81) | (N = 81) | (N = 85) | (N = 79) |
| FACIT- | 30.9% | 55.6% | 58.8% | 69.6% |
| fatigue | | | | |
| response | | | | |

| | | | |
|---|---|---|---|
| Difference from placebo | 24.7% | 28.0% | 38.8% |
| (95% CI) | (9.9, 39.5) | (13.4, 42.5) | (24.5, 53.0) |
| p-value | 0.002 | <0.001 | <0.001 |

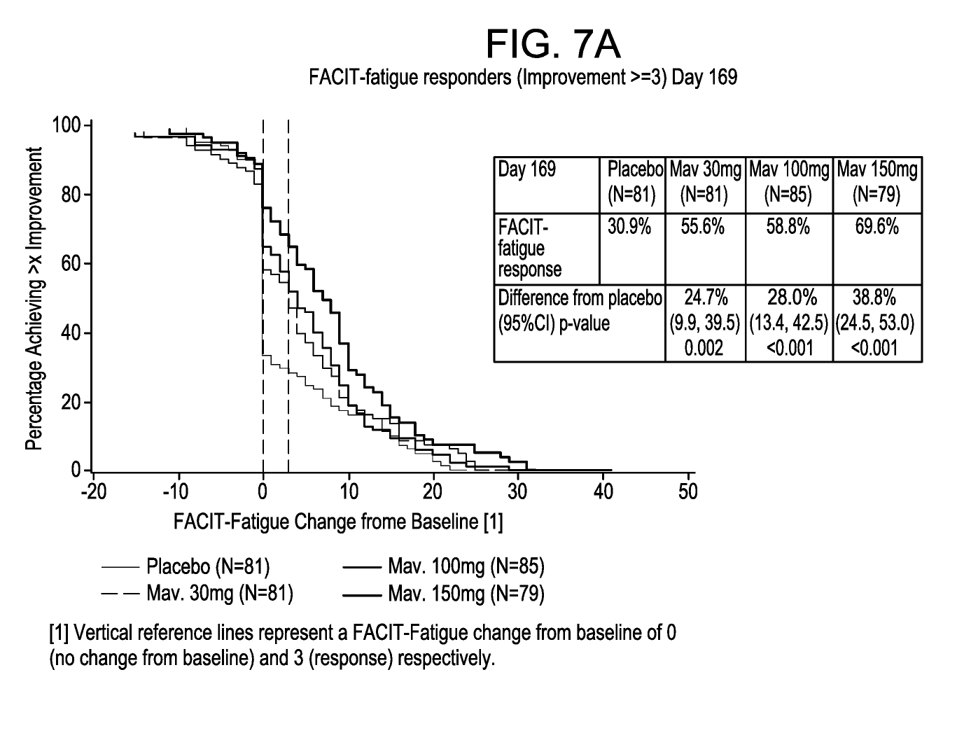

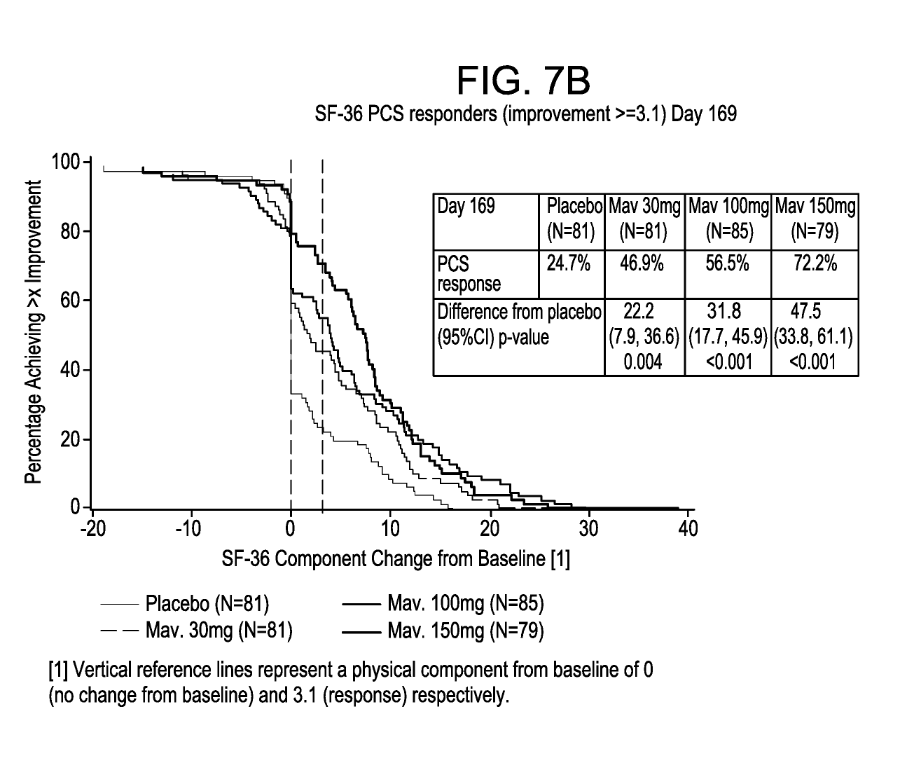

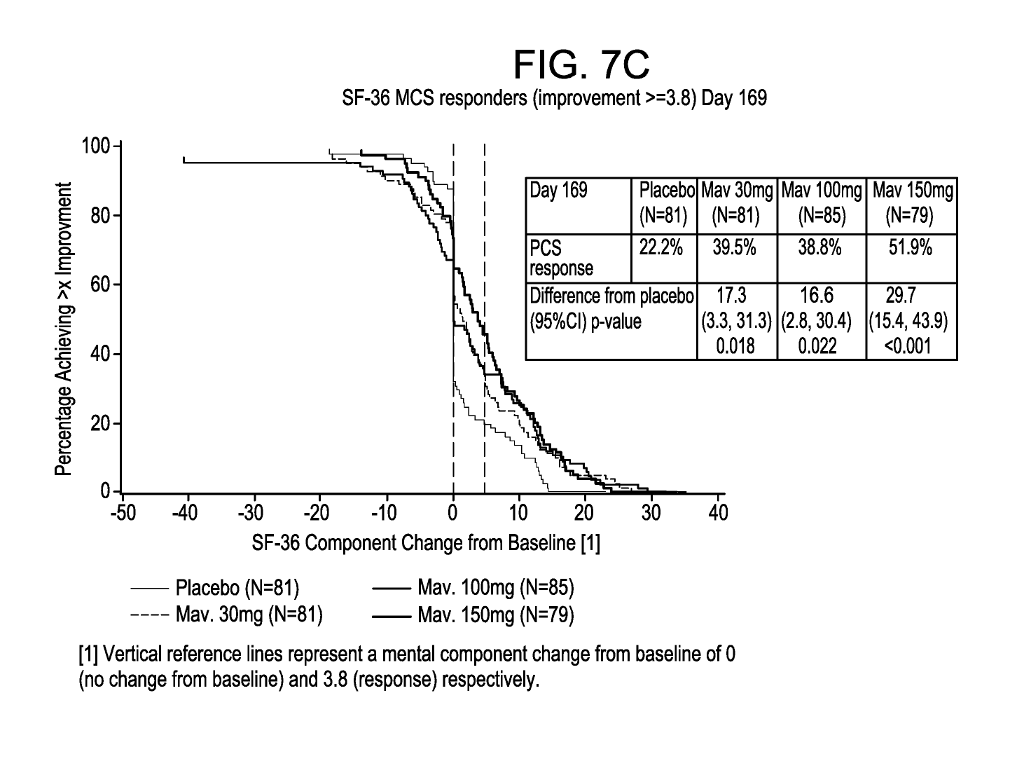

FIG. 7A is a graphical representation of Table 7B showing FACIT-fatigue responders (improvement>=3) at Day 169. The FACIT (Functional Assessment of Chronic Illness Therapy) measurement system is a collection of questionnaires targeted to the management of chronic illness. In addition to reporting improvement based on FACIT, patients experienced improvements in both physical and mental domain scores following administration of mavrilimumab as shown below. Table 7C shows the physical component score where an improvement of at least 3.1 is clinically important. Table 7D shows the mental component score where an improvement of at least 3.8 is clinically important. Table 7E shows SF-36® Physical Component Summary responders at Day 169. FIG. 7B is a graphical representation showing SF-36® PCS responders (improvement>=3.1) at Day 169.

TABLE 7C
Physical Component Score

| | Placebo | Mav 30 mg | Mav 100 mg | Mav 150 mg |
|---|---|---|---|---|
| Visit | (N = 81) | (N = 81) | (N = 85) | (N = 79) |
| Day 85 | 2.84 | 4.29 | 5.13 | 6.24 |
| Difference from placebo | | 1.45 | 2.29 | 3.40 |
| (95% CI) | | (−0.67, 3.57) | (0.20, 4.38) | (1.29, 5.52) |
| p-value | | 0.178 | 0.032 | 0.002 |
| Day 169 | 3.21 | 5.44 | 6.55 | 7.58 |

| | | | |
|---|---|---|---|
| Difference from placebo | 2.23 | 3.34 | 4.38 |
| (95% CI) | (−0.59, 5.06) | (0.58, 6.10) | (1.60, 7.15) |
| p-value | 0.120 | 0.018 | 0.002 |

TABLE 7D
Mental Component Score

| | Placebo | Mav 30 mg | Mav 100 mg | Mav 150 mg |
|---|---|---|---|---|
| Visit | (N = 81) | (N = 81) | (N = 85) | (N = 79) |
| Day 85 | 1.64 | 3.07 | 5.06 | 5.63 |
| Difference from placebo | | 1.43 | 3.42 | 3.99 |
| (95% CI) | | (−1.45, 4.31) | (0.57, 6.27) | (1.11, 6.87) |
| p-value | | 0.331 | 0.019 | 0.007 |
| Day 169 | 3.11 | 3.94 | 4.17 | 5.38 |

| | | | |
|---|---|---|---|
| Difference from placebo | 0.84 | 1.07 | 2.27 |
| (95% CI) | (−2.61, 4.28) | (−2.30, 4.43) | (−1.11, 5.65) |
| p-value | 0.633 | 0.534 | 0.187 |

TABLE 7E
SF-36 ® Physical Component Summary

| | | | | Mav |
|---|---|---|---|---|
| | Placebo | Mav 30 mg | Mav 100 mg | 150 mg |
| Day 169 | (N = 81) | (N = 81) | (N = 85) | (N = 79) |
| PCS response | 24.7% | 46.9% | 56.5% | 72.2% |

| | | | |
|---|---|---|---|
| Difference from | 22.2 | 31.8 | 47.5 |
| placebo (95% CI) | (7.9, 36.6) | (17.7, 45.9) | (33.8, 61.1) |
| p-value | 0.004 | <0.001 | <0.001 |

Table 7F shows SF-36® functional health mental component responders at Day 169. FIG. 7C is a graphical representation showing SF-36® Mental Component Summary (MCS) responders (improvement>=3.8) at Day 169.

TABLE 7F
SF-36 ® Mental Component Summary

| | | | | Mav |
|---|---|---|---|---|
| | Placebo | Mav 30 mg | Mav 100 mg | 150 mg |
| Day 169 | (N = 81) | (N = 81) | (N = 85) | (N = 79) |
| MCS response | 22.2% | 39.5% | 38.8% | 51.9% |

| | | | |
|---|---|---|---|
| Difference from placebo | 17.3 | 16.6 | 29.7 |
| (95% CI) | (3.3, 31.3) | (2.8, 30.4) | (15.4, 43.9) |
| p-value | 0.018 | 0.022 | <0.001 |

Table 7G depicts the Clinical Disease Activity Index (CDAI) response in patients treated with 150 mg doses of mavrilimumab. CDAI is patient focused and does not include CRP. It consists of swollen and tender joint count and the global disease score assessed by the patient and rheumatologist. A CDAI reduction of 6.5 represents moderate improvement. Table 7G, below shows that treatment with mavrilimumab resulted in better than moderate improvement at all doses and time points tested.

TABLE 7G
CDAI Response

| | Placebo | Mav 30 mg | Mav 100 mg | Mav 150 mg |
|---|---|---|---|---|
| Endpoint | (N = 81) | (N = 81) | (N = 85) | (N = 79) |
| CDAI Baseline | 40.9 | 42.4 | 42.9 | 40.5 |
| Day 85 | −9.1 (1.6) | −19.1 (1.5) | −20.8 (1.5) | −23.0 (1.6) |
| Adj mean | | | | |
| Change (SE) | | | | |
| Difference from placebo | | −10.0 | −11.7 | −13.9 |
| (95% CI) | | (−14.9, −5.7) | (−16.0, −7.4) | (−18.2, −9.5) |
| P-value | | <0.001 | <0.001 | <0.001 |
| Day 169 | −12.6 (1.8) | −22.0 (1.6) | −24.1 (1.5) | −26.1 (1.6) |
| Adj mean | | | | |
| Change (SE) | | | | |

| | | | |
|---|---|---|---|
| Difference from placebo | −9.4 | −11.5 | −13.5 |
| (95% CI) | (−14.2, −4.6) | (−16.3, −6.8) | (−18.3, −8.8) |
| P-value | <0.001 | <0.001 | <0.001 |

### Efficacy Summary

The results of the Phase 2b study show that the co-primary endpoints, DAS28 and ACR20, were highly statistically significant for all three doses. A clear and statistically significant dose response was observed for DAS28 and Day 85. Moreover the results were compared to standard treatments using a Demin longitudinal meta-analysis. As shown in FIG. 8, the ACR20 results observed in the current study, and in particular the results for the 150 mg cohort, were highly competitive in terms for speed of onset as well as the maximum ACR20 response rate.

### Example 3

### Safety

The incidence of adverse events was recorded for this study. The tables below summarize the adverse events (AEs) and serious adverse events (SAEs) that occurred while the patients were enrolled in the study with mavrilimumab. Table 8A provides an overview of adverse events. Table 8B provides a list of the most common adverse events (i.e. >3% on any dose) that occurred during the study. Table 8C highlights the adverse events of special interest. Table 8D lists the serious adverse events that were detected during the study. Table 8E lists the adverse events that led to withdrawal from the study.

TABLE 8A
Adverse Event Overview

| AE category | Placebo | Mav 30 mg | Mav 100 mg | Mav 150 mg |
|---|---|---|---|---|
| N (%) | (N = 81) | (N = 81) | (N = 85) | (N = 79) |
| Any AE | 38 (46.9%) | 41 (50.6%) | 36 (42.4%) | 43 (54.4%) |
| Any AE of | 11 (13.6%) | 7 (8.6%) | 5 (5.9%) | 11 (13.9%) |
| special interest | | | | |
| Any SAE | 1 (1.2%) | 4 (4.9%) | 5 (5.9%) | 2 (2.5%) |
| Any AE with | 0 | 0 | 0 | 0 |
| outcomeVdeath | | | | |
| Any AE | 2 (2.5%) | 3 (3.7%) | 3 (3.5%) | 4 (5.1%) |
| leading to | | | | |
| discontinuation | | | | |

TABLE 8B
Most Common Adverse Events

| AE preferred | Placebo | Mav 30 mg | Mav 100 mg | Mav 150 mg |
|---|---|---|---|---|
| term N (%) | (N = 81) | (N = 81) | (N = 85) | (N = 79) |
| Any AE | 38 (46.9%) | 41 (50.6%) | 36 (42.4%) | 43 (54.3%) |
| Headache | 2 (2.5%) | 5 (6.2%) | 4 (4.7%) | 6 (7.6%) |
| Naso- | 6 (7.4%) | 4 (4.9%) | 3 (3.5%) | 6 (7.6%) |
| pharyngitis | | | | |
| Hypertension | 2 (2.5%) | 4 (4.9%) | 4 (4.7%) | 3 (3.8%) |
| Bronchitis | 6 (7.4%) | 3 (3.7%) | 1 (1.2%) | 4 (5.1%) |
| Hyper- | 0 | 2 (2.5%) | 0 | 3 (3.8%) |
| lipidaemia | | | | |
| Influenza | 0 | 1 (1.2%) | 3 (3.5%) | 1 (1.3%) |
| Rheumatoid | 4 (4.9%) | 2 (2.5%) | 2 (2.4%) | 0 |
| Arthritis | | | | |
| Neutropenia | 1 (1.2%) | 0 | 0 | 3 (3.8%) |

TABLE 8C
Adverse Events of Special Interest

| | | Mav | | |
|---|---|---|---|---|
| AE preferred | Placebo | 30 mg | Mav 100 mg | Mav 150 mg |
| term N (%) | (N = 81) | (N = 81) | (N = 85) | (N = 79) |
| Any special | 11 (13.6%) | 7 (8.6%) | 5 (5.9%) | 11 (13.9%) |
| interest AE | | | | |
| Hepatic function | 0 | 0 | 1 (1.2%) | 0 |
| abnormalities* | | | | |
| Hypersensitivity | 2 (2.5%) | 2 (2.5%) | 0 | 3 (3.8%) |
| reactions | | | | |
| Serious infections | 0 | 1 (1.2%) | 0 | 0 |
| Malignancies | 0 | 0 | 1 (1.2%) | 1 (1.3%) |
| Neutropenia** | 1 (1.2%) | 0 | 0 | 2 (2.5%) |
| Pulmonary events | 8 (9.9%) | 5 (6.2%) | 3 (3.5%) | 5 (6.3%) |

*Hepatic function abnormalities = AST/ALT >3*ULN and bilirubin >2*ULN. One case of Hy's Law due to cholelithiasis
**Neutropenia = absolute neutrophil <1.0 10\hat{ }3/μL

TABLE 8D
Serious Adverse Events

| | | Mav | Mav | Mav |
|---|---|---|---|---|
| AE preferred term | Placebo | 30 mg | 100 mg | 150 mg |
| N (%) | (N = 81) | (N = 81) | (N = 85) | (N = 79) |
| Any SAE | 1 (1.2%) | 4 (4.9%) | 5 (5.9%) | 2 (2.5%) |
| Atrial tachycardia | 0 | 0 | 1 (1.2%) | 0 |
| Supraventricular | 0 | 0 | 1 (1.2%) | 0 |
| tachycardia | | | | |
| Dyspepsia | 0 | 0 | 1 (1.2%) | 0 |
| Cholelithiasis | 0 | 1 (1.2%) | 0 | 0 |
| Pneumonia | 0 | 1 (1.2%) | 0 | 0 |
| Lower limb fracture | 0 | 1 (1.2%) | 0 | 0 |
| Tendon rupture | 0 | 0 | 1 (1.2%) | 0 |
| Osteoarthritis | 0 | 1 (1.2%) | 0 | 0 |
| Rheumatoid arthritis | 1 (1.2%) | 0 | 0 | 0 |
| Adenocarcinoma of the | 0 | 0 | 1 (1.2%) | 0 |
| cervix | | | | |
| Squamous cell | 0 | 0 | 0 | 1 (1.3%) |
| carcinoma of lung | | | | |
| Cystocele | 0 | 1 (1.2%) | 0 | 0 |
| Angioedema | 0 | 0 | 0 | 1 (1.2%) |

TABLE 8E
Adverse Events Leading to Discontinuation

| | | Mav | Mav | Mav |
|---|---|---|---|---|
| AE preferred term | Placebo | 30 mg | 100 mg | 150 mg |
| N (%) | (N = 81) | (N = 81) | (N = 85) | (N = 79) |
| Any AE leading to | 2 (2.5%) | 3 (3.7%) | 3 (3.5%) | 4 (5.1%) |
| discontinuation | | | | |
| Neutropenia | 0 | 0 | 0 | 1 (1.3%) |
| Supraventricular | 0 | 0 | 1 (1.2%) | 0 |
| tachycardia | | | | |
| Drug hypersensitivity | 0 | 1 (1.2%) | 0 | 0 |
| Angioedema | 0 | 0 | 0 | 1 (1.2%) |
| Cellulitis | 0 | 0 | 0 | 1 (1.3%) |
| Pneumonia | 1 (1.2%) | 1 (1.2%) | 0 | 0 |
| Bronchiectasis | 0 | 0 | 1 (1.2%) | 0 |
| Osteoarthritis | 0 | 1 (1.2%) | 0 | 0 |
| Rheumatoid arthritis | 1 (1.2%) | 0 | 0 | 0 |
| Adenocarcinoma of | 0 | 0 | 1 (1.2%) | 0 |
| the cervix | | | | |
| Squamous cell | 0 | 0 | 0 | 1 (1.3%) |
| carcinoma of lung | | | | |

Pulmonary Function Tests (PFT) measure how well the lungs take in and release air, and how well they move gases such as oxygen from the atmosphere into the body's circulation. FEV is the Forced Expiratory Volume; FVC is the Forced Vital Capacity. Table 8F lists PFTs largest reduction from baseline during the study.

TABLE 8F
PFTs Largest Reduction From Baseline

| | | Placebo | | Mav 30 mg | | Mav 100 mg | | Mav 150 mg | |
|---|---|---|---|---|---|---|---|---|---|
| | | (N = 81) | | (N = 81) | | (N = 85) | | (N = 79) | |
| | n | 80 | | 81 | | 85 | | 79 | |
| FEV1 percent | <=15% | 78 | (97.5%) | 76 | (96.2%) | 74 | (89.2%) | 68 | (87.2%) |
| predicted | | | | | | | | | |
| | >15-20% | 1 | (1.3%) | 2 | (2.5%) | 4 | (4.8%) | 6 | (7.7%) |
| | >20% | 1 | (1.3%) | 1 | (1.3%) | 5 | (6.0%) | 4 | (5.1%) |
| FVC percent | <=15% | 77 | (96.3%) | 77 | (97.5%) | 74 | (89.2%) | 70 | (89.7%) |
| predicted | | | | | | | | | |
| | >15-20% | 0 | | 0 | | 3 | (3.6%) | 4 | (5.1%) |
| | >20% | 3 | (3.8%) | 2 | (2.5%) | 6 | (7.2%) | 4 | (5.1%) |

The laboratory values for neutrophils; alanine amino transferase (ALT) in U/L; aspartate aminotransferase (AST) in U/L; and LDL cholesterol in mg/dL were obtained at regular intervals throughout the six months of treatment. The laboratory values furthest from the norm during the six months are given below in Tables 8G to 8J. Table 8G lists the neutrophils levels (10\hat{0}3/μL); Table 8H lists the ALT levels; Table 8I lists the AST levels, and Table J lists the LDL cholesterol levels.

TABLE 8G
neutrophil levels

| Minimum | | | Mav | | Mav | | Mav | |
|---|---|---|---|---|---|---|---|---|
| post-baseline | Placebo | | 30 mg | | 100 mg | | 150 mg | |
| (10\hat{ }3/μL) | (N = 81) | | (N = 81) | | (N = 85) | | (N = 79) | |
| n | 80 | | 81 | | 85 | | 79 | |
| <0.5 | 0 | | 0 | | 0 | | 0 | |
| 0.5-<1 | 1 | (1.3%) | 0 | | 0 | | 2 | (2.5%) |
| 1-<1.5 | 1 | (1.3%) | 1 | (1.2%) | 2 | (2.4%) | 1 | (1.3%) |
| 1.5-<LLN | 3 | (3.8%) | 8 | (9.9%) | 11 | (12.9%) | 9 | (11.4%) |
| >=LLN | 75 | (93.8%) | 72 | (88.9%) | 72 | (84.7%) | 67 | (84.8%) |

TABLE 8H
alanine aminotransferase levels

| Maximum | | Mav | | Mav | | Mav | |
|---|---|---|---|---|---|---|---|
| post-baseline | Placebo | 30 mg | | 100 mg | | 150 mg | |
| (U/L) | (N = 81) | (N = 81) | | (N = 85) | | (N = 79) | |
| n | 80 | 81 | | 85 | | 79 | |
| <ULN | 56 (70.0%) | 53 | (65.4%) | 56 | (65.9%) | 47 | (59.5%) |
| >1-<=3xULN | 24 (30.0%) | 26 | (32.1%) | 24 | (28.2%) | 28 | (35.4%) |
| >3-<=5xULN | 0 | 2 | (2.5%) | 3 | (3.5%) | 4 | (5.1%) |

| | | | | | |
|---|---|---|---|---|---|
| >5-<=20xULN | 0 | 0 | 1 | (1.2%) | 0 |
| >20xULN | 0 | 0 | 1 | (1.2%) | 0 |

TABLE 8I
aspartate aminotransferase levels

| Maximum | | | Mav | | Mav | | Mav | |
|---|---|---|---|---|---|---|---|---|
| post-baseline | Placebo | | 30 mg | | 100 mg | | 150 mg | |
| (U/L) | (N = 81) | | (N = 81) | | (N = 85) | | (N = 79) | |
| n | 80 | | 81 | | 85 | | 79 | |
| <ULN | 66 | (82.5%) | 57 | (70.4%) | 64 | (75.3%) | 49 | (62.0%) |
| >1-<=3xULN | 14 | (17.5%) | 23 | (28.4%) | 16 | (18.8%) | 30 | (38.0%) |

| | | | | | | |
|---|---|---|---|---|---|---|
| >3-<=5xULN | 0 | 1 | (1.2%) | 3 | (3.5%) | 0 |

| | | | | | |
|---|---|---|---|---|---|
| >5-<=20xULN | 0 | 0 | 2 | (2.4%) | 0 |

| | | | | |
|---|---|---|---|---|
| >20xULN | 0 | 0 | 0 | 0 |

TABLE 8J
LDL cholesterol levels

| | | Placebo | | Mav 30 mg | | Mav 100 mg | | Mav 150 mg | |
|---|---|---|---|---|---|---|---|---|---|
| | | (N = 81) | | (N = 81) | | (N = 85) | | (N = 79) | |
| Baseline | N | 80 | | 81 | | 85 | | 78 | |
| | Mean (SD) | 118.1 | (42.2) | 116.9 | (32.1) | 116.1 | (38.0) | 122.2 | (40.6) |
| Day 57 | N | 77 | | 78 | | 85 | | 78 | |
| | Mean (SD) | 107.1 | (34.6) | 117.4 | (34.2) | 113.8 | (37.1) | 120.1 | (36.4) |
| Day 155 | N | 38 | | 63 | | 70 | | 69 | |
| | Mean (SD) | 109.2 | (43.3) | 123.2 | (37.4) | 116.2 | (34.2) | 123.2 | (39.4) |

### Safety Summary

Results of the trial indicated a similar percentage of pulmonary AEs on active and placebo. There were no cases of PAP or suggestive of PAP. Two pneumonia cases were reported: (i) placebo non-serious with pleural effusion and (ii) 30 mg serious infection. There were no other serious infections. There was one case of Hy's Law due to cholelithiasis but no other clinically meaningful laboratory abnormalities. No anaphylaxis. Two hypersensitivity AEs leading to discontinuation (drug hypersensitivity on 30 mg and angioedema on 150 mg).

### Example 4

### Study Design Overview: Japanese Study

The study was a randomized, double-blind, placebo-controlled, single-dose study to evaluate the pharmacokinetics, immunogenicity, and safety of mavrilimumab in healthy Japanese subjects. The primary objective was to evaluate pharmacokinetics (PK) and immunogenicity of single subcutaneous (SC) 100 mg and 150 mg doses of mavrilimumab in healthy adult Japanese subjects with a secondary objective to evaluate the safety of mavrilimumab following a single SC 100 mg and 150 mg does in healthy Japanese subjects. FIGS. 1A and 1B show the study design. The subjects were from age 20 to 55 and of Japanese ethnicity, i.e., both of the subjects' parents and both sets of grandparents being Japanese and the subjects having lived outside of Japan for 5 or less years. The Japanese subjects were in good physical and mental health with no evidence of clinical significant respiratory disease: DLCO, FEV1 & FVC greater than or equal to 70% predicted.

Table 9 shows the demographic breakdown of treatment of the subjects.

TABLE 9
Demographics

| | | | Mav | | Mav | |
|---|---|---|---|---|---|---|
| Charac- | Placebo | | 100 mg | | 150 mg | |
| teristic | (N = 4) | | (N = 10) | | (N = 10) | |
| Age | 29.8 | (21-35) | 32.1 | (22-43) | 29.5 | (20-39) |
| (years) | | | | | | |
| (range) | | | | | | |
| Female | 50% | | 70% | | 50% | |
| Male | 50% | | 30% | | 50% | |
| Asian | 100% | | 100% | | 100% | |
| Weight | 61.0 | (50-70) | 56.0 | (49-72) | 58.7 | (49-64) |
| (Kg) | | | | | | |
| (range) | | | | | | |
| BMI | 21.0 | (18.6-22.8) | 20.7 | (18.1-23.7) | 21.0 | (18.5-23.3) |
| (kg/m²) | | | | | | |
| (range) | | | | | | |

### Example 5

### Pharmacokinetics: Japanese Study

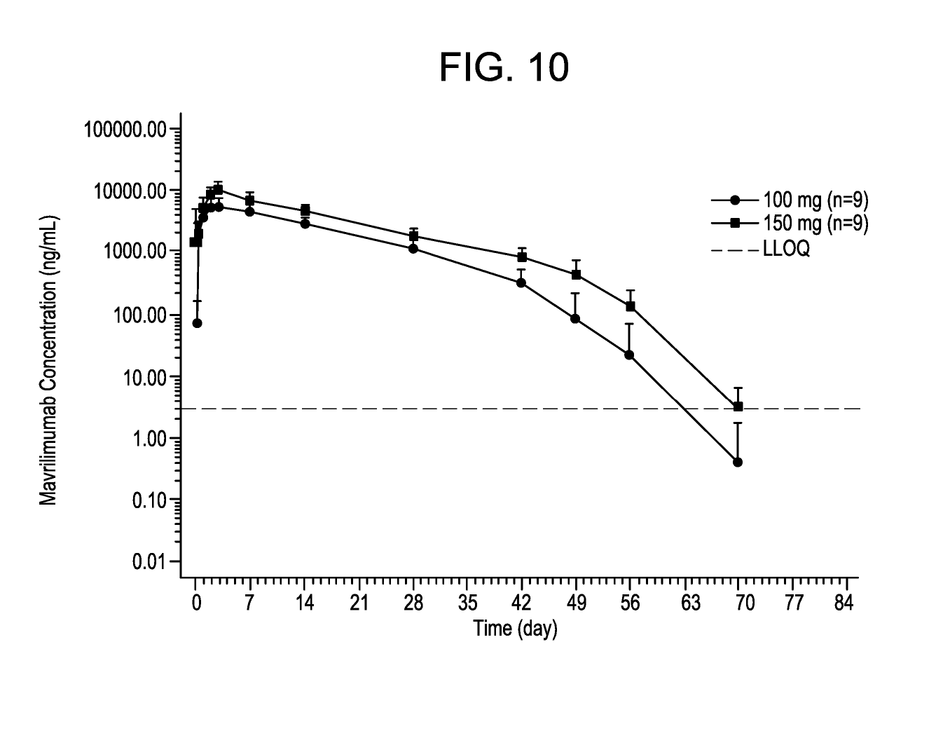

FIG. 10 shows the mean pharmacokinetic profiles of single SC mavrilimumab doses (100 mg and 150 mg) in healthy Japanese subjects to about 75 days. Table 10 shows non-compartmental analysis (NCA) results.

TABLE 10
PK Parameters-NCA Results

| | | tₘₐₓ | Cₘₐₓ | AUC∞ | CL/F | t½ |
|---|---|---|---|---|---|---|
| | N | (day) | (mg/mL) | (mg/mL × day) | (L/day) | (day) |
| 100 | 9 | 2 | 6.32 ± 3.61 | 95.1 ± 28.4 | 1.14 ± | 4.91 ± |
| mg | | (3-7) | | | 0.379 | 2.54 |
| 150 | 9 | 3 | 10.8 ± 2.91 | 157 ± 48.4 | 1.12 ± | 3.74 ± |
| mg | | (0.0833-7) | | | 0.669 | 1.32 |

There were 2 ADA+(anti-drug antibodies) samples (titer=1) on Day 85, 1 in each treatment arm (100 mg/150 mg) mavrilimumab. Data from these two subjects were excluded from Table 10. In total, 6.1% (17 of 280 samples) were excluded from the NCA.

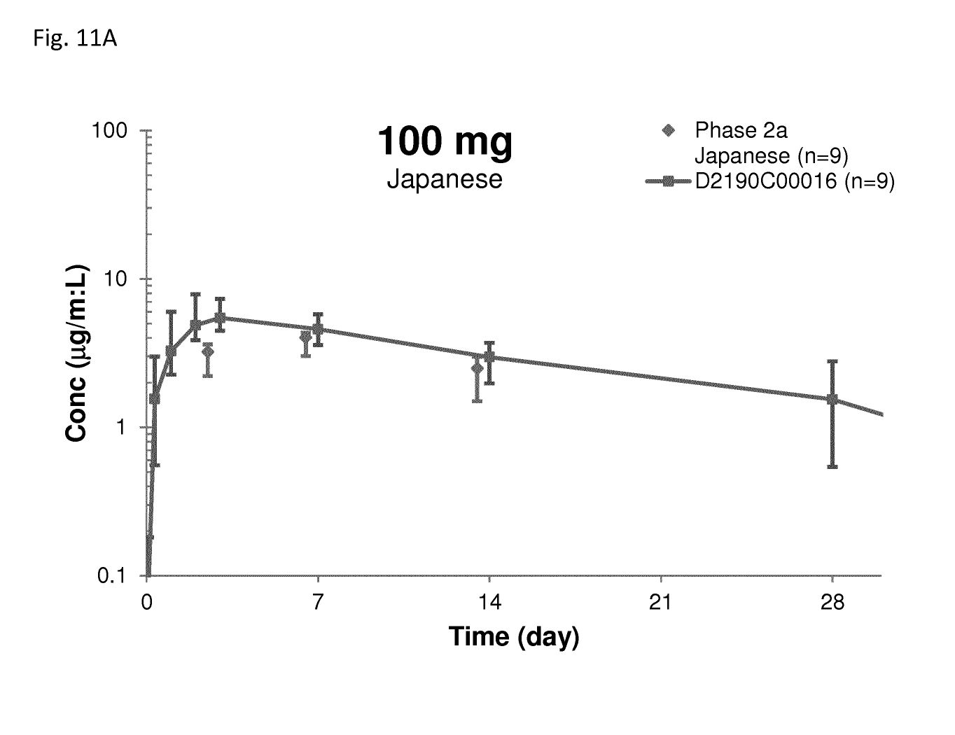

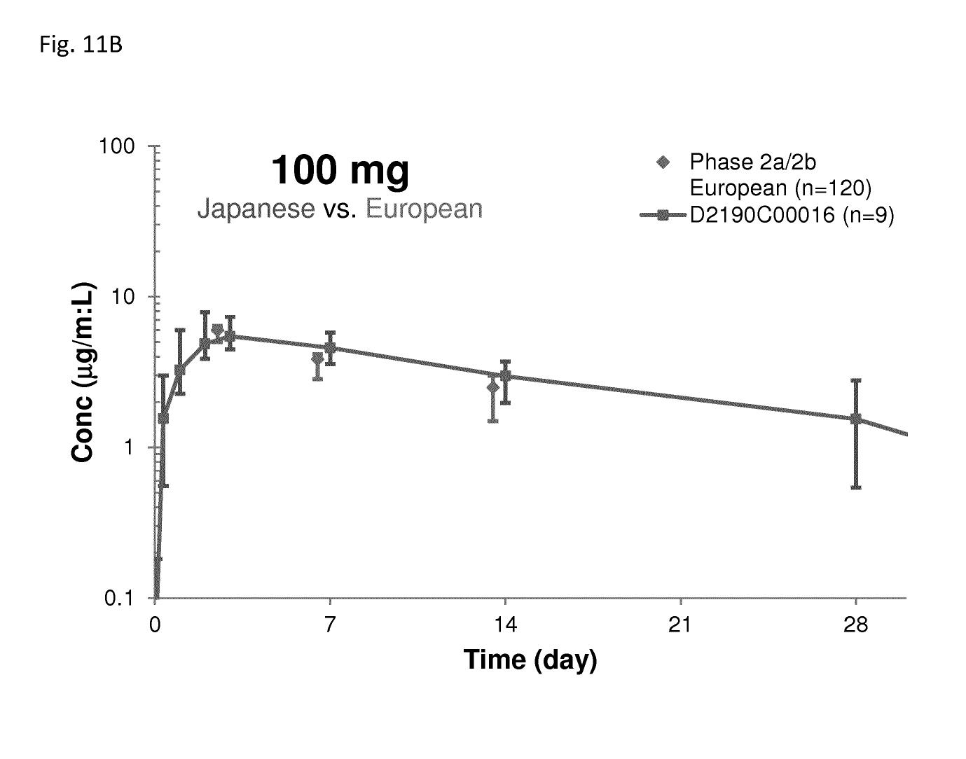

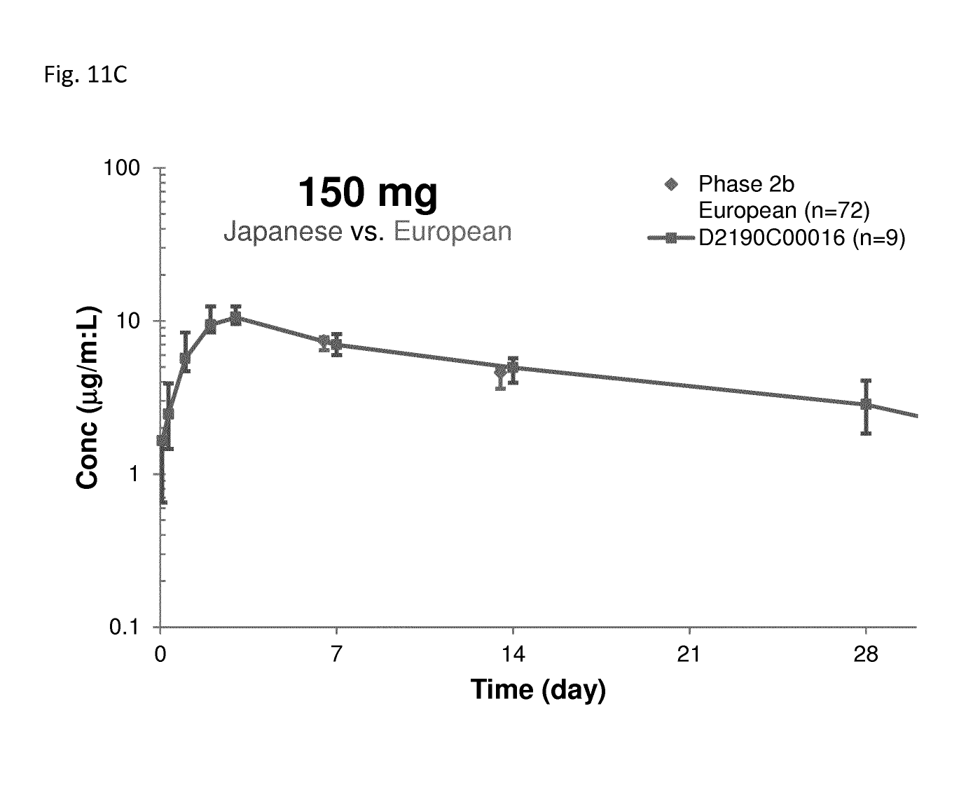

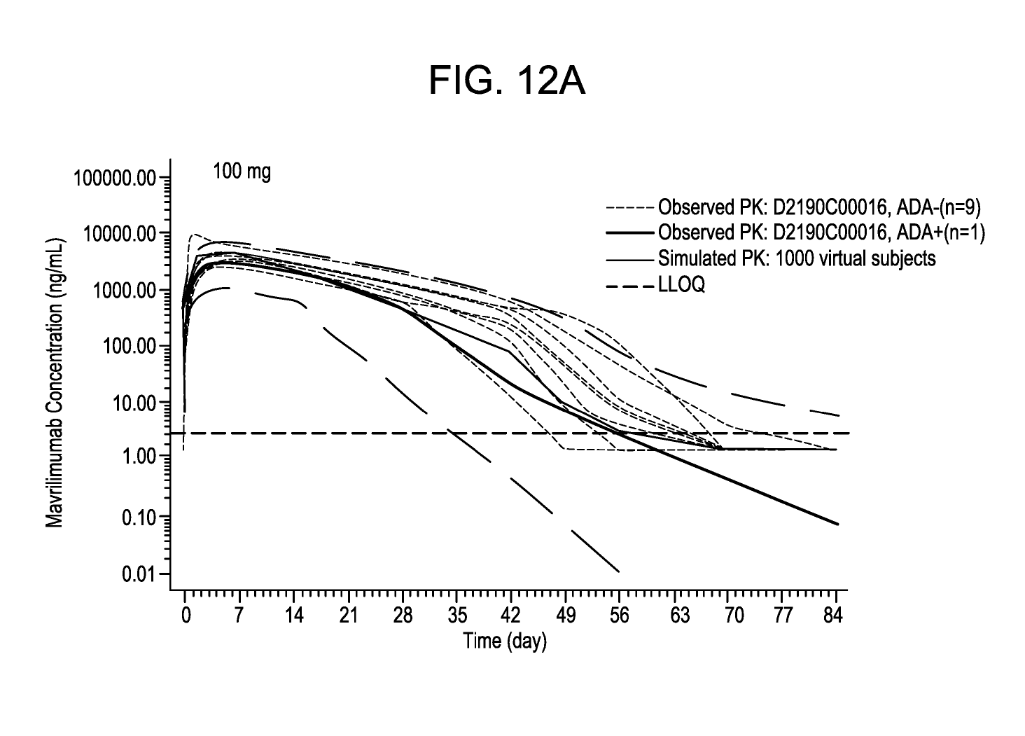

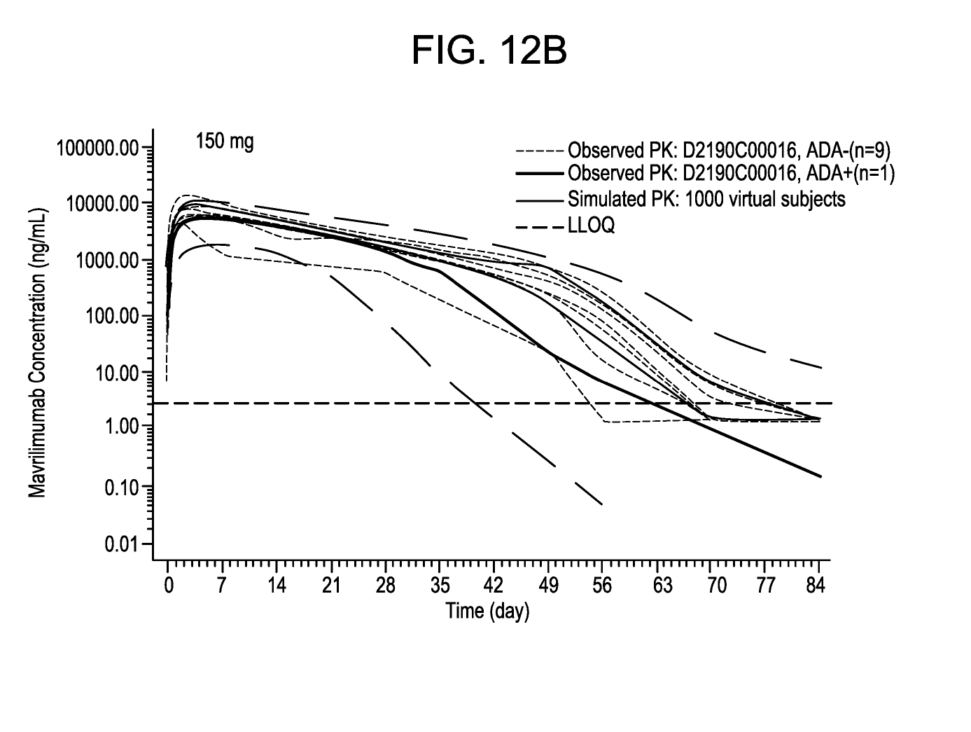

FIGS. 11A, 11B, and 11C show a cross-study comparison of pharmacokinetic results between the present Japanese study and separate trials of 100 mg and 150 mg mavrilimumab. The Japanese study data was consistent with previous studies. FIGS. 12A and 12B show that the observed PK profiles of the Japanese subjects fell within the predicted range (excluding ADA+ and outliers) at 100 mg and 150 mg mavrilimumab, respectively.

Table 11 further shows anti-drug antibody (ADA) data.

TABLE 11
ADA

| | Placebo | Mav 100 mg | Mav 150 mg |
|---|---|---|---|
| | (N = 4) | (N = 10) | (N = 10) |
| Baseline ADA n | 4 | 10 | 10 |
| ADA positive at baseline | 0 | 0 | 0 |
| Post baseline ADA n | 4 | 10 | 10 |
| ADA positive post-baseline | 0 | 1 (10%)¹ | 1 (10%)² |
| Persistent positive* | 0 | 1 (10%)¹ | 1 (10%)² |

¹Negative at baseline, Day 29 and 57. Positive on Day 85.
²Negative at baseline, Day 29 and 57. Positive on Day 85.
*Persistent positive is defined as positive at ≧2 post-baseline assessments (with ≧16 weeks between first and last positive) or positive at last post-baseline assessment.

### Example 6

### Safety: Japanese Study

The incidence of adverse events (AE) was recorded for this study. Tables 12A, 12B, and 12C summarize the adverse events that occurred while the subjects were enrolled in the study with mavrilimumab.

TABLE 12A
Overview of adverse events (AE)

| AE category | Placebo | Mav 100 mg | Mav 150 mg |
|---|---|---|---|
| N (%) | (N = 4) | (N = 10) | (N = 10) |
| Any AE | 2 (50%) | 5 (50%) | 4 (40%) |
| Any AE of special interest | 0 | 0 | 1 (10%)* |
| Any AE grade 3 or higher | 0 | 0 | 0 |
| Any SAE | 0 | 0 | 0 |
| Any AE with outcome death | 0 | 0 | 0 |
| Any AE leading to discontinuation | 0 | 0 | 0 |

*Drug hypersensitivity (see Table 14 - AEs of special interest slide for details)

TABLE 12B
Adverse events (AE) by frequency

| AE preferred term | Placebo | Mav 100 mg | Mav 150 mg |
|---|---|---|---|
| N (%) | (N = 4) | (N = 10) | (N = 10) |
| Any AE | 2 (50%) | 5 (50%) | 4 (40%) |
| Oropharyngeal pain | 1 (25%) | 0 | 2 (20%) |
| Rhinitis | 1 (25%) | 1 (10%) | 1 (10%) |
| Nasopharyngitis | 0 | 2 (20%) | 0 |
| Drug hypersensitivity | 0 | 0 | 1 (10%) |
| Dysmenorrhoea | 0 | 1 (10%) | 0 |
| Headache | 0 | 1 (10%) | 0 |
| Injection site reaction | 0 | 0 | 1 (10%) |
| Laceration | 0 | 1 (10%) | 0 |
| Nausea | 0 | 0 | 1 (10%) |
| Presyncope | 0 | 1 (10%) | 0 |
| Upper respiratory | 0 | 1 (10%) | 0 |
| tract infection | | | |

TABLE 12C
Adverse events (AE) of special interest

| AE preferred term | Placebo | Mav 100 mg | Mav 150 mg |
|---|---|---|---|
| N (%) | (N = 4) | (N = 10) | (N = 10) |
| Any special interest AE | 0 | 0 | 1 (10.0%) |
| Hepatic function | 0 | 0 | 0 |
| abnormalities¹ | | | |
| Hypersensitivity reactions | 0 | 0 | 1 (10%)* |
| Serious infections | 0 | 0 | 0 |
| Malignancies | 0 | 0 | 0 |
| Neutropenia² | 0 | 0 | 0 |
| Pulmonary events | 0 | 0 | 0 |

¹Hepatic function abnormalities = AST/ALT > 3*ULN and bilirubin > 2*ULN.
²Neutropenia = absolute neutrophil < 1.0 103/μL
*One subject reported a grade 2 (moderate) related AE of HYPERSENSITIVITY REACTION DUE TO INVESTIGATIONAL MEDICINAL PRODUCT (Verbatim Term) starting in the dosing day, ~4 h after injection. Site description: “the subject developed a widespread pruritic erythematous rash on face, neck, chest and limbs at 1200 h that fully resolved by 1430 h (without any treatment). The subject was subjectively feeling well, vital signs were normal.”

Pulmonary Function Tests (PFT) measure how well the lungs take in and release air, and how well they move gases such as oxygen form the atmosphere into the body's circulation. FEV is the Forced Expiratory Volume. FVC is the Forced Vital Capacity. Table 12D shows results from PFTs assessed at baseline and Day 85.

TABLE 12D
Pulmonary function tests at Day 85

| | | | Mav | Mav |
|---|---|---|---|---|
| Pulmonary | | Placebo | 100 mg | 150 mg |
| function test | Category | (N = 4) | (N = 10) | (N = 10) |
| FEV1 | N | 4 | 10 | 10 |
| (% predicted) | Baseline mean | 99.30% | 98.50% | 98.60% |
| | >15%-≦20 | 0 | 0 | 0 |
| | reduction | | | |
| | >20% reduction | 0 | 0 | 0 |
| FVC | N | 4 | 10 | 10 |
| (% predicted) | Baseline mean | 90.00% | 93.00% | 92.40% |
| | >15%-≦20 | 0 | 0 | 0 |
| | reduction | | | |
| | >20% reduction | 0 | 0 | 0 |
| DLCO | N | 4 | 10 | 10 |
| (% predicted) | Baseline mean | 90.50% | 86.10% | 78.80% |
| | >15%-≦20 | 0 | 1 (10%)¹ | 1 (10%)³ |
| | reduction | | | |
| | >20% reduction | 0 | 1 (10%)² | 0 |

¹Subject 11 Baseline = 86%; Day 85 = 70%, no adverse events
²Subject 05 Baseline = 89%; Day 85 = 68%, 1 AE: grade 1 rhinitis (Day 23-33)
³Subject 04 Baseline = 80%; Day 85 = 61%, 1 AE: grade 1 injection site reaction on Day 2

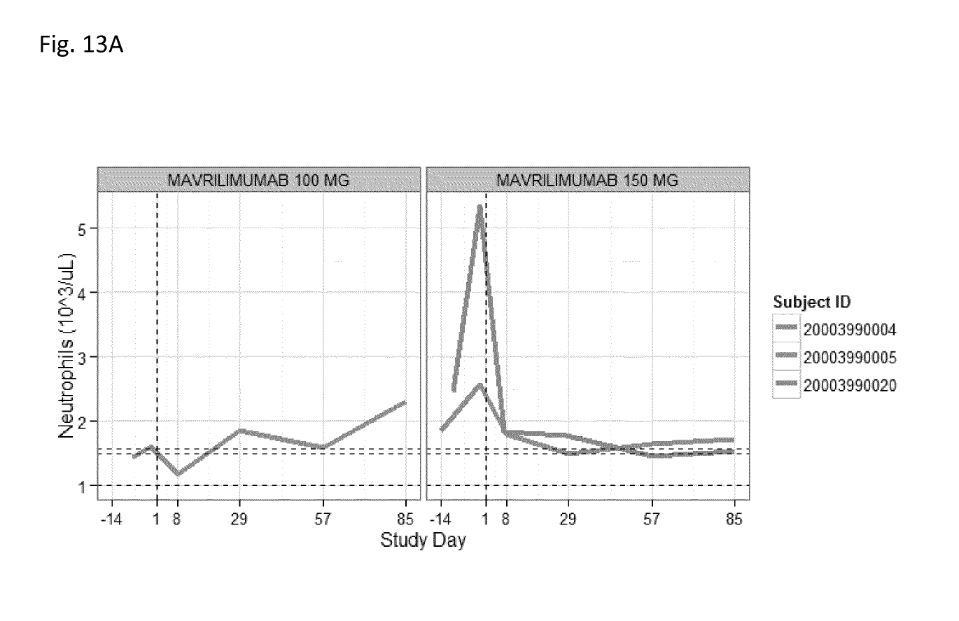

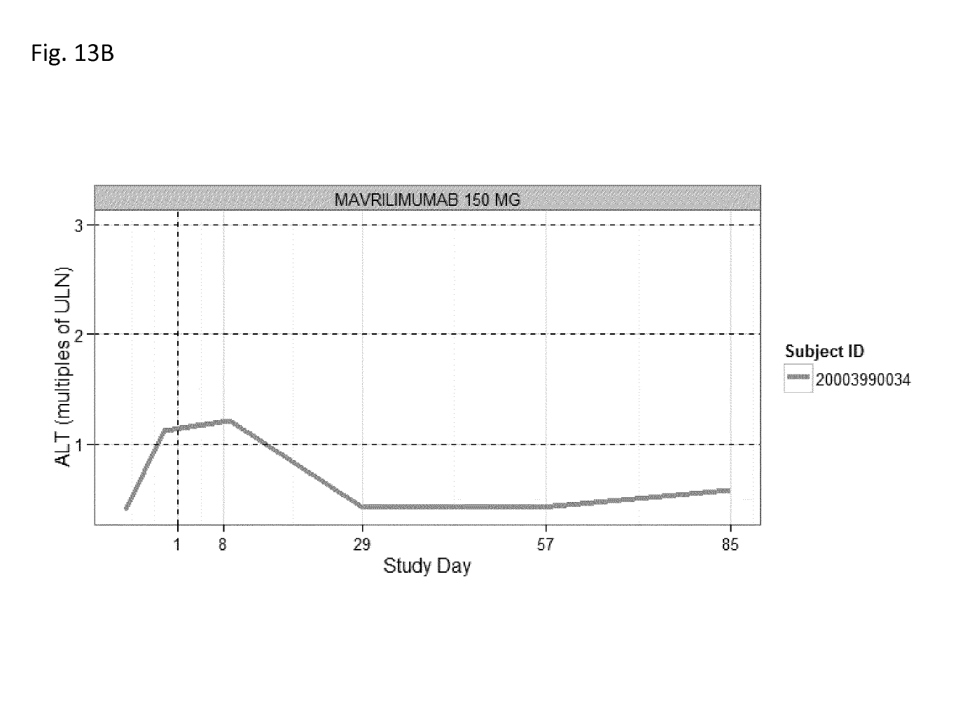

The laboratory values for neutrophils; alanine amino transferase (ALT) in U/L; and aspartate aminotransferase (AST) in U/L were obtained and are shown in Tables 12E, 12F, and 12G, respectively. Neutrophil profiles for subjects with a value less than lower limit of normal (LLN) are shown in FIG. 13A and ATL profiles for subjects with a value greater than upper limit of normal (ULN) are shown in FIG. 13B.

TABLE 12E
Neutrophils (10³/μL)

| | Placebo | Mav 100 mg | Mav 150 mg |
|---|---|---|---|
| Minimum post-baseline | (N = 4) | (N = 10) | (N = 10) |
| n | 4 | 10 | 10 |
| <0.5 | 0 | 0 | 0 |
| 0.5-<1 | 0 | 0 | 0 |
| 1-<1.5 | 0 | 1 (10%) | 1 (10%) |
| 1.5-<LLN | 0 | 0 | 1 (10%) |
| >=LLN | 4 (100%) | 9 (90%) | 8 (80%) |

TABLE 12F
ALT (U/L)

| | Placebo | Mav 100 mg | Mav 150 mg |
|---|---|---|---|
| Maximum post-baseline | (N = 4) | (N = 10) | (N = 10) |
| n | 4 | 10 | 10 |
| <ULN | 4 (100%) | 10 (100%) | 9 (90%) |
| >1-<=3xULN | 0 | 0 | 1 (10%) |
| >3-<=5xULN | 0 | 0 | 0 |
| >5-<=20xULN | 0 | 0 | 0 |
| >20xULN | 0 | 0 | 0 |

TABLE 12G
AST (U/L)

| | Placebo | Mav 100 mg | Mav 150 mg |
|---|---|---|---|
| Maximum post-baseline | (N = 4) | (N = 10) | (N = 10) |
| n | 4 | 10 | 10 |
| <ULN | 4 (100%) | 10 (100%) | 10 (100%) |
| >1-<=3xULN | 0 | 0 | 0 |
| >3-<=5xULN | 0 | 0 | 0 |
| >5-<=20xULN | 0 | 0 | 0 |
| >20xULN | 0 | 0 | 0 |

### Safety Summary

No overall safety signals were observed. The percentages of AEs were similar between active groups and placebo. No deaths, severe/life-threatening or serious AEs were reported. One moderate, related AESI of Drug Hypersensitivity (PT) occurred in the 150 mg mavrilimumab group, starting 4 h after dosing and being resolved within 3 hours without necessitating treatment.

There were few DLCO fluctuations or isolated values <70% of the predicted value, which were not considered as clinically significant by the PI and were not referred to pulmonologist.

The foregoing description of the specific embodiments will so fully reveal the general nature of the provided embodiments that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

What is claimed is:

1. A method of treating rheumatoid arthritis, comprising administering to a patient or a population of patients in need of treatment a composition comprising mavrilimumab, wherein the composition is administered at a dose of 150 mg mavrilimumab.

2. The method of claim 1, where mavrilimumab is administered every two weeks.

3. The method of claim 1 or 2, wherein mavrilimumab is administered by oral, intravenous, or subcutaneous administration.

4. The method of claim 1, wherein the Disease Activity Score 28 joint assessment which includes a measurement of C-Reactive Protein (DAS28-CRP) decreases from baseline by more than 1.7, more than 1.8, more than 1.9, more than 2.0, or more than 2.1 in one or more of the patients.

5. The method of any one of claims 1 to 4, wherein the 1987 American College of Rheumatology (ACR) criteria improves at least 20% treatment efficacy (ACR 20), at least 50% treatment efficacy (ACR50), or at least 70% treatment efficacy (ACR70) in one or more of the patients.

6. The method of any one of claims 1 to 4, wherein the decrease in DAS28-CRP from baseline is by more than 1.7, by more than 1.8, or by more than 1.9 in one or more of the patients within 169 days of initiation of treatment.

7. The method of any one of claims 1 to 4, wherein the decrease in DAS28-CRP from baseline is by more than 1.7, by more than 1.8, or by more than 1.9 in one or more of the patients within 85 days of initiation of treatment.

8. The method of any one of claims 1 to 4, wherein the decrease in DAS28-CRP from baseline is by more than 2.0 or by more than 2.1 in one or more treated patients within 169 days of initiation of treatment.

9. The method of any one of claims 1 to 4, which can achieve ACR 20 in one or more of the patients.

10. The method of claim 9, which can achieve ACR 20 in one or more of the patients within 160 days of initiation of treatment.

11. The method of claim 9, which can achieve ACR 20 in one or more of the patients within 80 days of initiation of treatment.

12. The method of claim 9, which can achieve ACR 20 in one or more of the patients within 40 days of initiation of treatment.

13. The method of claim 9, which can achieve ACR 20 in one or more of the patients within 14 days of initiation of treatment.

14. The method of any one of claims 1 to 4, which can achieve ACR 50 in one or more of the patients.

15. The method of claim 14, which can achieve ACR 50 in one or more of the patients within 160 days of initiation of treatment.

16. The method of claim 14, which can achieve ACR 50 in one or more of the patients within 80 days of initiation of treatment.

17. The method of claim 14, which can achieve ACR 50 in one or more of the patients within 42 days of initiation of treatment.

18. The method of claim 14, which can achieve ACR 50 in one or more of the patients within 14 days of initiation of treatment.

19. The method of any one of claims 1 to 4, which can achieve ACR 70 in one or more of the patients.

20. The method of claim 19, which can achieve ACR 70 in one or more of the patients within 160 days of initiation of treatment.

21. The method of claim 19, which can achieve ACR 70 in one or more of the patients within 80 days of initiation of treatment.

22. The method of claim 19, which can achieve ACR 70 in one or more of the patients within 40 days of initiation of treatment.

23. The method of claim 19, which can achieve ACR 70 in one or more of the patients within 14 days of initiation of treatment.

24. The method according to any of the preceding claims, which can result in remission or reduced time to onset of remission of rheumatoid arthritis symptoms in one or more of the patients.

25. The method of claim 24, which can result in remission of rheumatoid arthritis symptoms in at least 10%, at least 15%, or at least 20% of the patients.

26. The method of claim 9, which can achieve ACR 20 in at least 60% or at least 65% of the patients within 113 days.

27. The method of claim 14, which can achieve ACR 50 in at least 15%, at least 20%, or at least 25% of the patients within 113 days.

28. The method of claim 19, which can achieve ACR 70 in at least 10%, or at least 13% of the patients within 113 days.

29. The method of any of claims 24 to 26, which can achieve ACR20, ACR50, or ACR70 within 80 days of initiation of treatment in one or more of the patients.

30. The method according to any of the preceding claims, which can improve physical function in one or more of the patients, as determined by the Health Assessment Questionnaire Disability Index (HAQ-DI).

31. A method of improving physical function of an RA patient or a population of RA patients, as determined by a HAQ-DI score, comprising administering to a patient or population of patients in need of treatment a composition comprising mavrilimumab, wherein the composition is administered at a dose of 150 mg.

32. The method of claim 30 or claim 31, which can improve the HAQ-DI score by at least 0.25 in one or more of the patients.

33. The method of claim 32, which can improve the HAQ-DI score by at least 0.25 in at least 40%, or at least 60% of the patients.

34. The method of claim 32 or claim 33, wherein the improvement in HAQ-DI is achieved within four weeks, or within six weeks of initiation of treatment.

35. A method of inducing remission of RA in a patient or a population of patients as measured by a DAS28-CRP of less than 2.6, comprising administering to a patient or a population of patients in need of treatment a composition comprising mavrilimumab, wherein the composition is administered at a dose of 150 mg.

36. The method of claim 35, wherein the onset of remission is achieved within 160 days of initiation of treatment.

37. The method of claim 35, wherein the onset of remission is achieved within 80 days of initiation of treatment.

38. The method of claim 35, wherein the onset of remission is achieved within 30 days, or within 42 days of initiation of treatment.

39. The method of claim 35, wherein the onset of remission is achieved within 14 days of initiation of treatment.

40. The method according to any of the preceding claims, further comprising administering an additional therapeutic agent.

41. The method of claim 40, wherein the additional therapeutic agent comprises a disease modifying anti-rheumatic drug (DMARD).

42. The method of claim 41, wherein DMARD is methotrexate.

43. The method of claim 42, wherein the methotrexate is administered at a dose of 7.5 to 25 mg per week.

44. The method of claim 42 or claim 43, wherein a stable dose of methotrexate is administered for at least 4 weeks prior to administration of the composition comprising mavrilimumab.

45. The method of any one of claims 42 to 44, comprising administering the composition comprising mavrilimumab to the patient in combination with continued doses of methotrexate.

46. The method according to any of the preceding claims, wherein one or more of the patients has a baseline DAS28-CRP of at least 3.2 prior to treatment.

47. The method of claim 46, wherein one or more of the patients has a baseline DAS28-CRP greater than 5.1 prior to treatment.

48. The method according to any of the preceding claims, wherein one or more of the patients tests positive for rheumatoid factor and/or anti-cyclic citrullinated peptide (CCP) IgG antibodies prior to treatment.

49. The method according to any of the preceding claims, wherein one or more of the patients does not have medically significant respiratory disease.

50. A composition comprising 150 mg of mavrilimumab suitable for oral administration, intravenous administration, or subcutaneous injection, for use in a method according to any of the preceding claims.

51. The composition of claim 50, formulated for administration in combination with methotrexate.

52. A method of treating rheumatoid arthritis comprising administering to a patient or a population of patients in need of treatment a composition comprising mavrilimumab, wherein the composition is administered at a dose of 150 mg mavrilimumab, and wherein the treatment results in an increase in duration of DAS28-CRP remission.

53. The method of claim 52, wherein the duration of DAS28-CRP remission is achieved within 60 days in at least 50%, or at least 60% of patients.

54. The method of claim 53, wherein the duration of DAS28-CRP remission is achieved within 80 days in at least 40%, or at least 50% of patients.

55. The method of claim 54, where in the duration of DAS28-CRP remission is achieved within 130 days in at least 20%, or at least 25%, or at least 30% of the patients.
